(19) United States
(12) Patent Application Publication
(10) Pub. No.: US 2025/0387246 A1
(43) Pub. Date: Dec. 25, 2025

(54) EXPANDABLE STENT DEVICES AND SYSTEMS

(71) Applicant: EDWARDS LIFESCIENCES CORPORATION, Irvine, CA (US)

(72) Inventors: Abdorruhman M. Yousef, Santa Ana, CA (US); Adeeb Saiduddin, Lake Forest, CA (US); Hannah Ariel Gibson Gerondale, Huntington Beach, CA (US); Andrew Paul Schaffer, Orange, CA (US); Alison Louise Rodriguez, Irvine, CA (US); Rachel Christine Leacock, Torrance, CA (US); Anthony Michael Romero, Corona, CA (US); Christine Marie Heyninck-Jantz, Trabuco Canyon, CA (US); Radhika Vaid, Rancho Mission Viejo, CA (US); Venkateswaran Shanmugam, Lake Forest, CA (US); Annie Man Yee Ratanapinta, Santa Ana, CA (US)

(21) Appl. No.: 19/313,406

(22) Filed: Aug. 28, 2025

Related U.S. Application Data
(63) Continuation of application No. PCT/US2024/018318, filed on Mar. 4, 2024.
(60) Provisional application No. 63/488,504, filed on Mar. 5, 2023.
(60) Provisional application No. 63/611,372, filed on Dec. 18, 2023.

(51) Int. Cl. A61F 2/90

(52) U.S. Cl. CPC A61F 2/90; A61F 2210/0004; A61F 2210/0057; A61F 2210/0066; A61F 2210/0071; A61F 2210/0076; A61F 2230/0004; A61F 2230/001; A61F 2250/0039; A61F 2250/0098

(57) ABSTRACT

Expandable frames for docking an expandable medical device can include a plurality of struts defining a plurality of cells. The expandable frame has an hourglass shaped profile when in an expanded condition, with flared endmost retaining portions, convex medial sealing portions, and a concave central waist portion. The convex medial sealing portions have a substantially uniform axially extending rounded contour in profile extending from the concave central waist portion to the flared endmost retaining portions.

### RELATED APPLICATION

The present application is a continuation of Patent Cooperation Treaty application no. PCT/US2024/018318, filed on Mar. 4, 2024, which claims priority to and all benefit of U.S. Provisional Patent Application Ser. No. 63/488,504, filed on Mar. 5, 2023, for EXPANDABLE STENT DEVICES AND SYSTEMS, and U.S. Provisional Patent Application Ser. No. 63/611,372, filed on Dec. 18, 2023, for EXPANDABLE STENT DEVICES AND SYSTEMS, the disclosures of both of which are incorporated herein by reference in their entirety.

### TECHNICAL FIELD

The present disclosure relates to expandable stents and, in particular, docking station stents, delivery systems, and methods for use in implanting a heart valve, e.g., a transcatheter heart valve (“THV”).

### BACKGROUND

Prosthetic heart valves can be used to treat cardiac valvular disorders. The native heart valves (the aortic, pulmonary, tricuspid, and mitral valves) serve critical functions in assuring the forward flow of an adequate supply of blood through the cardiovascular system. These heart valves can be rendered less effective by congenital, inflammatory, or infectious conditions. Such conditions can eventually lead to serious cardiovascular compromise or death. For many years, the definitive treatment for such disorders was the surgical repair or replacement of the valve during open heart surgery.

A transcatheter technique can also be used for introducing and implanting a prosthetic heart valve using a flexible catheter in a manner that is less invasive than open heart surgery. In this technique, a prosthetic valve can be mounted in a crimped state on the end portion of a flexible catheter and advanced through a blood vessel of the patient until the valve reaches the implantation site. The valve at the catheter tip can then be expanded to its functional size at the site of the defective native valve, such as by inflating a balloon on which the valve is mounted. Alternatively, the valve can have a resilient, self-expanding stent or frame that expands the valve to its functional size when it is advanced from a delivery sheath at the distal end of the catheter.

Transcatheter heart valves (THVs) may be appropriately sized to be placed inside most native aortic valves. However, with larger native valves, blood vessels, and grafts, aortic transcatheter valves might be too small to secure into the larger implantation or deployment site. In this case, the transcatheter valve may not be large enough to sufficiently expand inside the native valve or other implantation or deployment site to be secured in place.

Replacing the pulmonary valve, which is sometimes referred to as the pulmonic valve, presents significant challenges. The geometry of the pulmonary artery can vary greatly from patient to patient. Typically, the pulmonary artery outflow tract after corrective surgery is too wide for effective placement of a prosthetic heart valve.

### SUMMARY

This summary is meant to provide examples and is not intended to be limiting of the scope of the disclosure in any way. For example, any feature included in an example of this summary is not required by the claims, unless the claims explicitly recite the feature. The description discloses examples of expandable stents for implantable medical devices. The expandable stents can be constructed in a variety of ways.

In some examples, an expandable stent includes an expandable frame extending axially from a proximal end to a distal end and a material attached to an exterior of the expandable frame to define an outer periphery of the expandable stent.

In some examples, an expandable stent includes an expandable frame extending axially from a proximal end to a distal end and a material attached to an interior of the expandable frame to define an inner periphery of the expandable stent.

In some examples, an expandable stent includes an expandable frame extending axially from a proximal end to a distal end, an outer material attached to an exterior of the expandable frame to define an outer periphery of the expandable stent, and an inner material attached to an interior of the expandable frame to define an inner periphery of the expandable stent.

In some examples, the outer material comprises a permeable material and the inner material comprises an impermeable material.

In some examples, at least a portion of the outer material defines a tissue engaging retaining portion.

In some examples, at least a portion of the inner material defines a valve seat.

In some examples, at least a portion of the outer material is axially aligned with at least a portion of the inner material.

In some examples, the axially aligned portions of the first and inner materials encapsulate at least a portion of the expandable frame.

In some examples, at least a portion of the outer material is directly attached to the expandable frame.

In some examples, the at least a portion of the outer material is attached to the expandable frame by an adhesive.

In some examples, the at least a portion of the outer material is sutured to the expandable frame.

In some examples, the at least a portion of the outer material is attached to the expandable frame through heat fixation.

In some examples, at least a portion of the inner material is directly attached to the expandable frame.

In some examples, the at least a portion of the inner material is attached to the expandable frame by an adhesive.

In some examples, the at least a portion of the inner material is sutured to the expandable frame.

In some examples, the at least a portion of the inner material is attached to the expandable frame through heat fixation.

In some examples, at least a portion of the outer material is directly attached to at least a portion of the inner material.

In some examples, the at least a portion of the outer material is attached to the at least a portion of the inner material by an adhesive.

In some examples, the at least a portion of the outer material is sutured to the at least a portion of the inner material.

In some examples, the at least a portion of the outer material is attached to the at least a portion of the inner material through heat fixation.

In some examples, the outer material comprises a shrink-wrap material.

In some examples, the inner material comprises a shrink-wrap material.

In some examples, the outer material comprises at least one of a knitted material, a woven material, and a perforated material.

In some examples, the outer material comprises at least one of polyethylene terephthalate (PET), fluorinated ethylene propylene (FEP), polytetrafluoroethylene (PTFE), ethylene tetrafluoroethylene (ETFE), and thermoplastic polyurethane (TPU).

In some examples, the inner material comprises at least one of polyethylene terephthalate (PET), fluorinated ethylene propylene (FEP), polytetrafluoroethylene (PTFE), ethylene tetrafluoroethylene (ETFE), and thermoplastic polyurethane (TPU).

In some examples, the expandable frame comprises a plurality of struts.

In some examples, the plurality of struts defines axially endmost apices, wherein the outer material covers exterior surfaces of the axially endmost apices.

In some examples, the plurality of struts defines a plurality of cells, wherein a portion of the plurality of cells is at least partially uncovered by the inner material.

In some examples, the portion of the plurality of cells is at least partially uncovered by both the outer material and the inner material.

In some examples, the at least partially uncovered portion of the plurality of cells is defined by strut portions that are covered by the outer material.

In some examples, the at least partially uncovered portion of the plurality of cells comprises distalmost cells of the plurality of cells.

In some examples, the expandable stent further comprises at least one radiopaque marker encapsulated between the first and inner materials.

In some examples, at least a portion of the inner material defines a valve seat, and wherein the at least one radiopaque marker is axially aligned with the valve seat.

In some examples, the inner material comprises first and second layers, wherein at least a portion of the first layer defines a valve seat.

In some examples, the expandable frame comprises a self-expanding frame.

In some examples, the expandable stent is a docking station, wherein the inner periphery is configured to retain an expandable medical device.

In some examples, the expandable stent is a docking station, wherein the inner periphery is configured to retain an expandable prosthetic valve.

In some examples, the inner material has a thickness between about 0.1 mm and about 0.15 mm.

In some examples, the outer material comprises a biodegradable or bioresorbable polymeric material.

In some examples, the biodegradable or bioresorbable polymeric material comprises at least one of poly-l-lactic acid (PLLA), polycaprolactone (PCL), and poly(4-hydroxybutyrate) (P4HB).

In some examples, the outer material has a thickness between about 0.05 mm and about 0.07 mm.

In some examples, the outer material comprises a woven material formed from yarns having a linear density between about 20 decitex and about 25 decitex.

In some examples, the outer material comprises a woven material formed from yarns having a thread density between about 160 picks per inch and about 225 picks per inch.

In some examples, the outer material has a thickness between about 0.2 mm and about 0.6 mm.

In some examples, the outer material is configured to experience a reduction in thickness of up to between about 15% and about 20% under compression in a deployed condition of the expandable stent.

In some examples, the outer material has a stretchability between about 40% and about 60%.

In some examples, the outer material comprises at least one of a knitted polymer material, a polymer foam, and a nonwoven textile material.

In some examples, the outer material comprises at least one of carbonate based, biocompatible, unmodified siloxinated, modified siloxinated, and fluorinated thermoplastic polyurethane (TPU).

In some examples, the outer material has a thickness between about 0.07 mm and about 0.20 mm.

In some examples, the outer material comprises a bidirectionally stretchable cloth material.

In some examples, the bidirectionally stretchable cloth material comprises honeycomb cloth.

In some examples, the outer material comprises a textured crochet knit material.

In some examples, the outer material has a thickness between about 0.40 mm and about 0.60 mm.

In some examples, the outer material has a stretchability between about 60% and about 100% in both an axial direction and a circumferential direction.

In some examples, an expandable frame comprises a plurality of struts defining a plurality of cells, the expandable frame having an hourglass shaped profile when in an expanded condition, with flared endmost retaining portions, convex medial sealing portions, and a concave central waist portion, wherein the convex medial sealing portions have a substantially uniform axially extending rounded contour in profile extending from the concave waist portion to the flared retaining portions.

In some examples, the sealing portions and the flared retaining portions are joined at inflection points positioned at a maximum diameter of the sealing portions when the frame is in the fully expanded condition, such that there are no radially inward extending portions between the sealing portions and the retaining portions.

In some examples, the sealing portions have a uniform radius of curvature of approximately 11.5 mm.

In some examples, the expandable frame further comprises a polymer coating applied to the plurality of struts at least at the sealing portions.

In some examples, the polymer coating comprises at least one of parylene and thermoplastic polyurethane (TPU).

In some examples, the polymer coating has a hardness between about 75 Shore A and about 90 Shore A.

In some examples, the polymer coating has a thickness between about 0.5 micron and about 4.0 micron.

In some examples, an expandable frame comprises a plurality of struts defining a plurality of cells, and a polymer coating applied to at least a radially outermost sealing portion of the plurality of struts.

In some examples, the polymer coating comprises at least one of parylene and thermoplastic polyurethane (TPU).

In some examples, the polymer coating has a hardness between about 75 Shore A and about 90 Shore A.

In some examples, the polymer coating has a thickness between about 0.5 micron and about 4.0 micron.

In some examples, a prosthetic valve assembly includes the expandable stent and a prosthetic valve secured to a valve seat of the expandable stent.

In some examples, the inner material sealingly engages the prosthetic valve.

In some examples, the prosthetic valve includes an expandable frame.

In some examples, a system includes a catheter including a sleeve, and the expandable stent, disposed in the sleeve in an unexpanded condition.

In some examples, in a method of manufacturing an expandable stent, an expandable frame is provided, extending axially from a proximal end to a distal end, and a material is attached to an exterior of the expandable frame to define an outer periphery of the expandable stent.

In some examples, in a method of manufacturing an expandable stent, an expandable frame is provided, extending axially from a proximal end to a distal end, and a material is attached to an interior of the expandable frame to define an inner periphery of the expandable stent.

In some examples, in a method of manufacturing an expandable stent, an expandable frame is provided, extending axially from a proximal end to a distal end, an outer material is attached to an exterior of the expandable frame to define an outer periphery of the expandable stent, and an inner material is attached to an interior of the expandable frame to define an inner periphery of the expandable stent.

In some examples, the outer material comprises a permeable material and the inner material comprises an impermeable material.

In some examples, at least a portion of the outer material defines a tissue engaging retaining portion.

In some examples, at least a portion of the inner material defines a valve seat.

In some examples, attaching the first and inner materials to the expandable frame comprises axially aligned at least a portion of the outer material with at least a portion of the inner material.

In some examples, attaching the first and inner materials to the expandable frame comprises encapsulating at least a portion of the expandable frame between axially aligned portions of the first and inner materials.

In some examples, attaching the outer material to the exterior of the expandable frame comprises directly attaching at least a portion of the outer material to the expandable frame.

In some examples, directly attaching the at least a portion of the outer material to the expandable frame comprises attaching the at least a portion of the outer material to the expandable frame by an adhesive.

In some examples, directly attaching the at least a portion of the outer material to the expandable frame comprises suturing the at least a portion of the outer material to the expandable frame.

In some examples, directly attaching the at least a portion of the outer material to the expandable frame comprises heat fixing the at least a portion of the outer material to the expandable frame.

In some examples, attaching the inner material to the interior of the expandable frame comprises directly attaching at least a portion of the inner material to the expandable frame.

In some examples, directly attaching the at least a portion of the inner material to the expandable frame comprises attaching the at least a portion of the inner material to the expandable frame by an adhesive.

In some examples, directly attaching the at least a portion of the inner material to the expandable frame comprises suturing the at least a portion of the inner material to the expandable frame.

In some examples, directly attaching the at least a portion of the inner material to the expandable frame comprises heat fixing the at least a portion of the inner material to the expandable frame.

In some examples, attaching the first and inner materials to the expandable frame comprises directly attaching at least a portion of the outer material to at least a portion of the inner material.

In some examples, directly attaching the at least a portion of the outer material to the at least a portion of the inner material comprises attaching the at least a portion of the outer material to the at least a portion of the inner material by an adhesive.

In some examples, directly attaching the at least a portion of the outer material to the at least a portion of the inner material comprises suturing the at least a portion of the outer material to the at least a portion of the inner material.

In some examples, directly attaching the at least a portion of the outer material to the at least a portion of the inner material comprises heat fixing the at least a portion of the outer material to the at least a portion of the inner material.

In some examples, the outer material comprises a tube of shrink-wrap material, wherein attaching the outer material to the exterior of the expandable frame comprises sliding the tube of shrink-wrap material over the expandable frame and shrinking the tube of shrink-wrap material against the exterior of the expandable frame.

In some examples, the inner material comprises a tube of shrink-wrap material, wherein attaching the inner material to the interior of the expandable frame comprises sliding the tube of shrink-wrap material into the expandable frame and expanding the tube of shrink-wrap material against the interior of the expandable frame.

In some examples, the outer material is thermoformed to a contoured shape corresponding to an exterior contoured surface of the expandable frame.

In some examples, the inner material is thermoformed to a contoured shape corresponding to an interior contoured surface of the expandable frame.

In some examples, attaching the outer material to the exterior of the expandable frame comprises wrapping a layer of the outer material around the exterior of the expandable frame.

In some examples, attaching the inner material to the interior of the expandable frame comprises wrapping a layer of the outer material around the interior of the expandable frame.

In some examples, attaching the outer material to the exterior of the expandable frame comprises covering exterior surfaces of axially endmost apices of the expandable frame with the outer material.

In some examples, the expandable frame comprises a plurality of struts defining a plurality of cells, wherein attaching the inner material to the interior of the expandable frame comprises leaving a portion of the plurality of cells at least partially uncovered by the inner material.

In some examples, attaching the outer material to the exterior of the expandable frame comprises leaving the portion of the plurality of cells at least partially uncovered by both the outer material and the inner material.

In some examples, the at least partially uncovered portion of the plurality of cells is defined by strut portions that are covered by the outer material.

In some examples, the at least partially uncovered portion of the plurality of cells comprises distalmost cells of the plurality of cells.

In some examples, at least one radiopaque marker is encapsulated between the first and inner materials.

In some examples, at least a portion of the inner material defines a valve seat, wherein the at least one radiopaque marker is axially aligned with the valve seat.

In some examples, the inner material comprises first and second layers, wherein at least a portion of the first layer defines a valve seat.

In some examples, the expandable frame comprises a self-expanding frame.

Various features as described elsewhere in this disclosure can be included in the examples summarized here and various methods and steps for using the examples and features can be used, including as described elsewhere herein.

Further understanding of the nature and advantages of the disclosed disclosures can be obtained from the following description and claims, particularly when considered in conjunction with the accompanying drawings in which like parts bear like reference numerals.

### BRIEF DESCRIPTION OF THE DRAWINGS

To further clarify various aspects of examples of the present disclosure, a more particular description of the certain examples will be made by reference to various aspects of the appended drawings. It is appreciated that these drawings depict only some examples of the present disclosure and are therefore not to be considered limiting of the scope of the disclosure. Moreover, while the figures may be drawn to scale for some examples, the figures are not necessarily drawn to scale for all examples. Examples of the present disclosure will be described and explained with additional specificity and detail through the use of the accompanying drawings.

FIG. 1A is a cutaway view of the human heart in a diastolic phase;

FIG. 1B is a cutaway view of the human heart in a systolic phase;

FIGS. 2A and 2B are sectional views of pulmonary arteries illustrating that pulmonary arteries can have a variety of different shapes and sizes;

FIGS. 3A and 3B are perspective views of pulmonary arteries illustrating that pulmonary arteries can have a variety of different shapes and sizes;

FIG. 4A is a schematic illustration of a compressed docking station being positioned in a circulatory system;

FIG. 4B is a schematic illustration of the docking station of FIG. 4A expanded to set the position of the docking station in the circulatory system;

FIG. 4C is a schematic illustration of an expandable transcatheter heart valve being positioned in the docking station illustrated by FIG. 4B;

FIG. 4D is a schematic illustration of the transcatheter heart valve of FIG. 4C expanded to set the position of the heart valve in the docking station;

FIG. 5A is a cutaway view of the human heart in a systolic phase with a docking station deployed in a pulmonary artery;

FIG. 5B is a cutaway view of the human heart in a systolic phase with a docking station and transcatheter heart valve deployed in a pulmonary artery;

FIG. 6A is an enlarged schematic illustration of the docking station and transcatheter heart valve of FIG. 5B when the heart is in the systolic phase;

FIG. 6B is a view taken in the direction indicated by lines 6B-6B in FIG. 6A;

FIG. 7 is a cutaway view of the human heart in a diastolic phase with a docking station and transcatheter heart valve deployed in a pulmonary artery;

FIG. 8A is an enlarged schematic illustration of the docking station and transcatheter heart valve of FIG. 7 when the heart is in the diastolic phase;

FIG. 8B is a view taken in the direction indicated by lines 8B-8B in FIG. 8A;

FIGS. 9A-9C are schematic cross-sectional side views of some examples of a docking station including a frame having an outer material attached to an exterior of the frame;

FIGS. 9D-9F are schematic cross-sectional side views of some examples of a docking station including a frame having an inner material attached to an interior of the frame;

FIGS. 9G-9L are schematic cross-sectional side views of some examples of a docking station including a frame having an outer material attached to an exterior of the frame and an inner material attached to an interior of the frame;

FIG. 10A is a schematic partial side view of a stent including outer and inner materials secured with a frame;

FIG. 10B is a schematic partial cross-sectional end view of the stent of FIG. 10A;

FIG. 11A is a schematic partial side view of a stent including outer and inner materials secured with a frame;

FIG. 11B is a schematic partial cross-sectional end view of the stent of FIG. 11A;

FIG. 12A is a schematic partial side view of a stent including outer and inner materials secured with a frame;

FIG. 12B is a schematic partial cross-sectional end view of the stent of FIG. 12A;

FIG. 13A is a schematic partial side view of a stent including outer and inner materials secured with a frame;

FIG. 13B is a schematic partial cross-sectional end view of the stent of FIG. 13A;

FIG. 14A is a schematic partial side view of a stent including outer and inner materials secured with a frame;

FIG. 14B is a schematic partial cross-sectional end view of the stent of FIG. 14A;

FIG. 15A is a schematic partial side view of a stent including outer and inner materials secured with a frame;

FIG. 15B is a schematic partial cross-sectional end view of the stent of FIG. 15A;

FIG. 16A is a perspective view of a stent including a frame aligned with tubular outer and inner materials;

FIG. 16B is a perspective view of the stent of FIG. 16A, with the outer and inner materials heat shrunk/stretched into close conformance with the frame;

FIG. 17A is a side view of an exemplary stent having an hourglass shaped profile with flared end portions;

FIG. 17B is a side profile of the stent of FIG. 17A;

FIG. 18A is a side view of an exemplary stent having an hourglass shaped profile with convex end portions;

FIG. 18B is a side profile of the stent of FIG. 18A;

FIG. 19A is a side view of an exemplary stent having a concave profile extending from flared end portions to a narrower waist portion;

FIG. 19B is a side profile of the stent of FIG. 19A;

FIG. 20A is a side view of an exemplary stent having a cylindrical shaped outer frame portion and a radially inward offset seat portion;

FIG. 20B is a side profile of the stent of FIG. 20A;

FIG. 21A is a side view of an exemplary stent having a cylindrical shaped outer frame portion and a radially inward and axially outward offset seat portion;

FIG. 21B is a side profile of the stent of FIG. 21A;

FIG. 22A is a side view of an exemplary stent having a radially outward extending flexible flanged portion extending from one end of an elongated cylindrical portion;

FIG. 22B is a side profile of the stent of FIG. 22A;

FIG. 23A is a side view of an exemplary stent having radially outward extending flexible flanged portions extending from both ends of an elongated cylindrical portion;

FIG. 23B is a side profile of the stent of FIG. 23A;

FIG. 24 is a side view of an exemplary stent having a cushioning exterior cover material;

FIG. 25 is a side view of another exemplary stent having a cushioning exterior cover material;

FIG. 26 is a side view of another exemplary stent having a cushioning exterior cover material;

FIG. 27 is a side view of another exemplary stent having a cushioning exterior cover material;

FIG. 28 is a side view of an exemplary stent having convex sealing portions configured to provide an extended tissue engaging surface portion;

FIG. 28A is a side profile view of the stent of FIG. 28;

FIG. 28B is a schematic illustration of the stent of FIG. 28 in a deployed condition;

FIGS. 29-32, and 33A-33C illustrate examples of valve types that can be deployed in a docking station, e.g., one of the docking stations described or depicted herein;

FIG. 34A is a cutaway view of the human heart in a systolic phase with a docking station being deployed in a pulmonary artery;

FIG. 34B is a cutaway view of the human heart in a systolic phase with a docking station deployed in a pulmonary artery;

FIG. 34C is a cutaway view of the human heart in a systolic phase with a docking station deployed in a pulmonary artery and a transcatheter heart valve being deployed in the pulmonary artery; and

FIG. 34D is a cutaway view of the human heart in a systolic phase with a docking station deployed in a pulmonary artery and a transcatheter heart valve being deployed in the pulmonary artery.

### DETAILED DESCRIPTION

The following description refers to the accompanying drawings, which illustrate some examples of the disclosure. Other examples having different structures and operation do not depart from the scope of the present disclosure. Some examples of the present disclosure are directed to devices and methods for providing a docking station or landing zone for a transcatheter heart valve (“THV”). In some examples, docking stations for THVs are illustrated as being used within the pulmonary artery, although the docking stations can be used in other areas of the anatomy, heart, or vasculature, such as the superior vena cava or the inferior vena cava. Further, the techniques and methods herein can be performed on a living animal or on a simulation, such as on a cadaver, cadaver heart, simulator (e.g., with the body parts, heart, tissue, etc. being simulated), etc. The docking stations described herein can be configured to compensate for the deployed THV being smaller than the space (e.g., anatomy/vasculature, etc.) in which it is to be placed.

It should be noted that various examples of docking stations and systems for delivery and implant are disclosed herein, and any combination of these options can be made unless specifically excluded. For example, any of the docking stations devices disclosed, can be used with any type of valve, and/or any delivery system, even if a specific combination is not explicitly described. Likewise, the different constructions of docking stations and valves can be mixed and matched, such as by combining any docking station type/feature, valve type/feature, tissue cover, etc., even if not explicitly disclosed. In short, individual components of the disclosed systems can be combined unless mutually exclusive or otherwise physically impossible.

For the sake of uniformity, in these figures and others in the application the docking stations are depicted such that the pulmonary bifurcation end is up, while the ventricular end is down. These directions may also be referred to as “distal” as a synonym for up or the pulmonary bifurcation end, and “proximal” as a synonym for down or the ventricular end, which are terms relative to the physician's perspective.

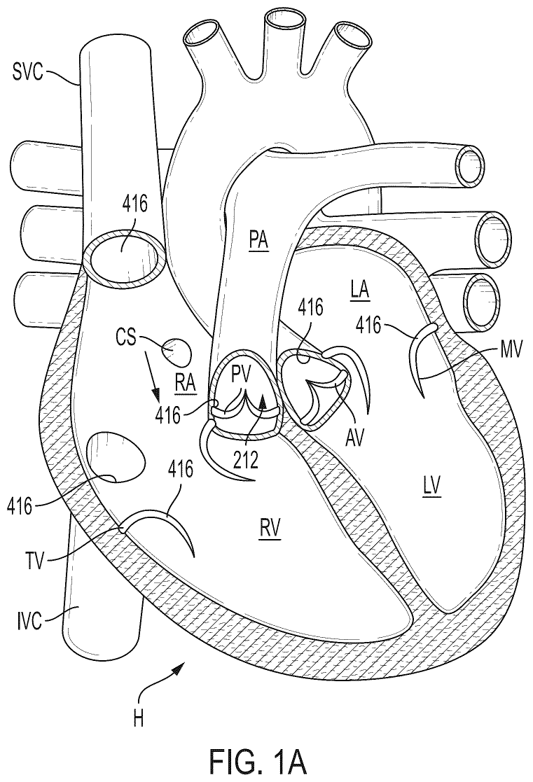

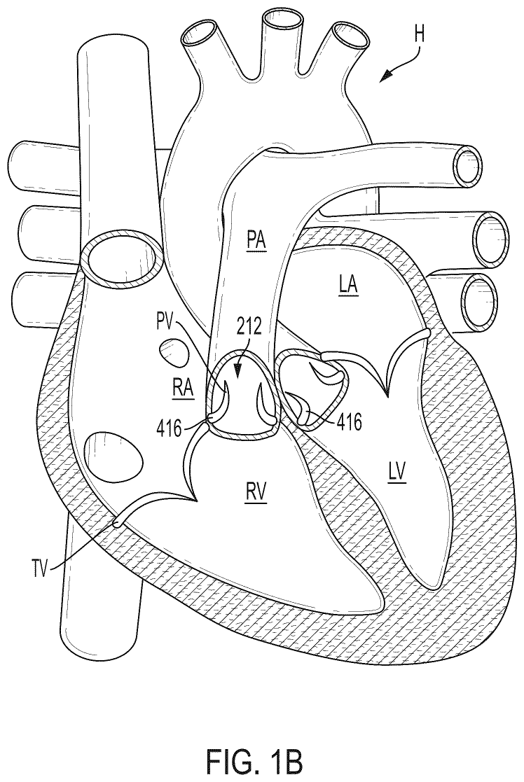

FIGS. 1A and 1B are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta (not identified) and the pulmonary valve PV separates the right ventricle from the pulmonary artery PA. The pulmonary valve PV is disposed at the inlet or start 212 of the pulmonary artery PA. Each of these valves has flexible leaflets extending inward across the respective orifices that come together or “coapt” in the flowstream to form the one-way, fluid-occluding surfaces. The docking stations and valves of the present application are described primarily with respect to the pulmonary valve. Therefore, anatomical structures of the right atrium RA and right ventricle RV will be explained in greater detail. It should be understood that the devices described herein can also be used in other areas, e.g., in the inferior vena cava IVC and/or the superior vena cava SVC as treatment for a regurgitant or otherwise defective tri-cuspid valve, in the aorta (e.g., an enlarged aorta) as treatment for a defective aortic valve AV, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The coronary sinus CS is a collection of veins joined together to form a large vessel that collects deoxygenated blood from the heart muscle (myocardium), and delivers it to the right atrium RA. During the diastolic phase, or diastole, seen in FIG. 1A, the venous blood that collects in the right atrium RA enters the tricuspid valve TV by expansion of the right ventricle RV. In the systolic phase, or systole, seen in FIG. 1B, the right ventricle RV contracts to force the venous blood through the pulmonary valve PV and pulmonary artery PA into the lungs. In some examples, the devices described by the present application are used to replace or supplement the function of a defective pulmonary valve PV. During systole, the leaflets of the tricuspid valve TV close to prevent the venous blood from regurgitating back into the right atrium RA.

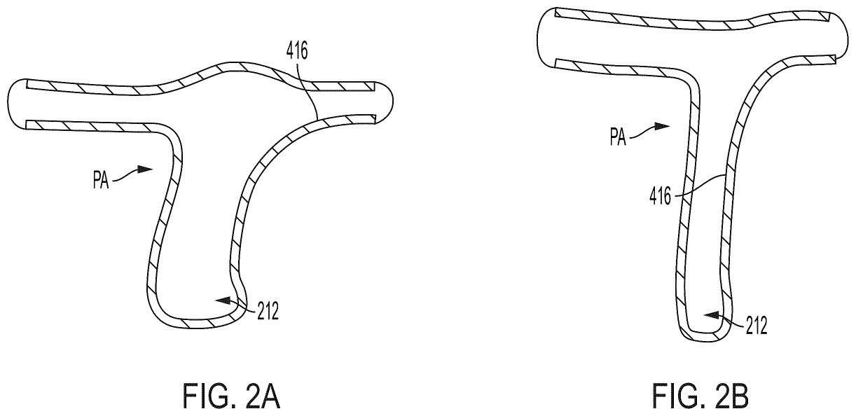

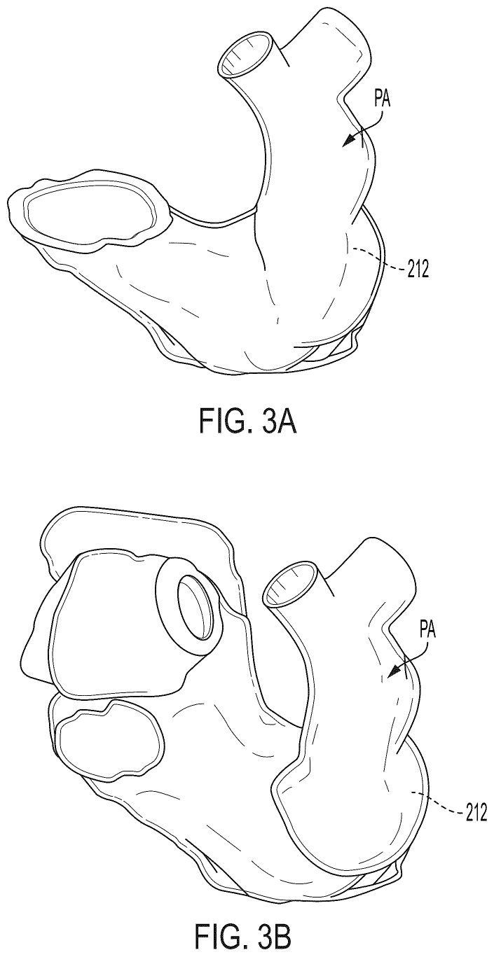

Referring to FIGS. 2A-2B and 3A-3B, the shown, non-exhaustive examples illustrate that the pulmonary artery can have a wide variety of different shapes and sizes. For example, as shown in the sectional views of FIGS. 2A and 2B and the perspective views of FIGS. 3A and 3B, the length, diameter, and curvature or contour may vary greatly between pulmonary arteries of different patients. Further, the diameter may vary significantly along the length of an individual pulmonary artery. These differences can be even more significant in pulmonary arteries that suffer from certain conditions and/or have been compromised by previous surgery. For example, the treatment of Tetralogy of Fallot (TOF) or Transposition of the Great Arteries (TGA) often results in larger and more irregularly shaped pulmonary arteries.

Tetralogy of Fallot (TOF) is a cardiac anomaly that refers to a combination of four related heart defects that commonly occur together. The four defects are ventricular septal defect (VSD), overriding aorta (the aortic valve is enlarged and appears to arise from both the left and right ventricles instead of the left ventricle as in normal hearts), pulmonary stenosis (narrowing of the pulmonary valve and outflow tract or area below the valve that creates an obstruction of blood flow from the right ventricle to the pulmonary artery), and right ventricular hypertrophy (thickening of the muscular walls of the right ventricle, which occurs because the right ventricle is pumping at high pressure).

Transposition of the Great Arteries (TGA) refers to an anomaly where the aorta and the pulmonary artery are “transposed” from their normal position so that the aorta arises from the right ventricle and the pulmonary artery from the left ventricle.

Surgical treatment for some conditions involves a longitudinal incision along the pulmonary artery, up to and along one of the pulmonary branches. This incision can eliminate or significantly impair the function of the pulmonary valve. A trans-annular patch is used to cover the incision after the surgery. The trans-annular patch reduces stenotic or constrained conditions of the pulmonary artery PA, associated with other surgeries. However, the impairment or elimination of the pulmonary valve PV can create significant regurgitation and, prior to the present disclosure, often required later open-heart surgery to replace the pulmonary valve. The trans-annular patch technique can result in pulmonary arteries having a wide degree of variation in size and shape (see, e.g., FIGS. 3A and 3B).

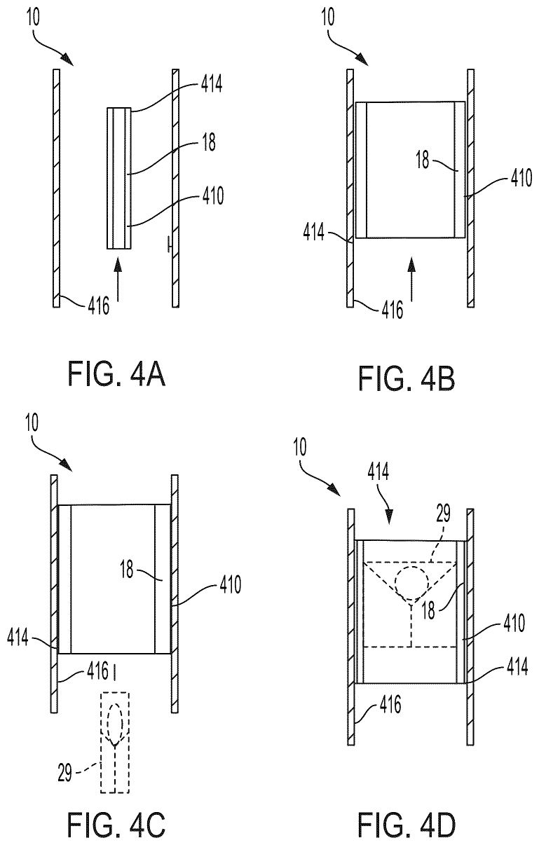

Referring to FIGS. 4A-4D, in some examples, an expandable docking station 10 includes one or more sealing portions 410, a valve seat 18, and one or more retaining portions 414. The sealing portion(s) 410 provide a seal between the docking station 10 and an interior surface 416 of the circulatory system. The valve seat 18 provides a supporting surface for implanting or deploying a valve 29 in the docking station 10 after the docking station 10 is implanted in the circulatory system. The retaining portions 414 help retain the docking station 10 and the valve 29 at the implantation position or deployment site in the circulatory system. Expandable docking station 10 and valve 29 as described in the various examples herein are also representative of a variety of docking stations and/or valves that might be known or developed, e.g., a variety of different types of valves could be substituted for and/or used as valve 29 in the various docking stations.

FIGS. 4A-4D schematically illustrate an exemplary deployment of the docking station 10 and valve 29 in the circulatory system. Referring to FIG. 4A, the docking station 10 is in a compressed form/configuration and is introduced to a deployment site in the circulatory system. For example, the docking station 10, can be positioned at a deployment site in a pulmonary artery by a catheter (e.g., one or more of the catheters described in co-owned U.S. Patent Application Publication No. 2019/0000615 and U.S. Pat. No. 10,363,130, the entire disclosures of both of which are incorporated herein by reference). Referring to FIG. 4B, the docking station 10 is expanded in the circulatory system such that the sealing portion(s) 410 and the retaining portions 414 engage the inside surface 416 of a portion of the circulatory system. Referring to FIG. 4C, after the docking station 10 is deployed, the valve 29 is in a compressed form and is introduced into the valve seat 18 of the docking station. Referring to FIG. 4D, the valve 29 is expanded in the docking station 10, such that the valve engages the valve seat 18. In the examples depicted herein, the docking station 10 is longer than the valve 29. However, in some examples the docking station 10 can be the same length or shorter than the length of the valve 29. Similarly, the valve seat 18 can be longer, shorter, or the same length as the length of the valve 29.

Referring to FIG. 4D, the valve 29 has expanded such that the seat 18 of the docking station supports the valve. The docking station 10 allows the valve 29 to operate within the expansion diameter range for which it is designed.

In some examples, the docking station 10 is configured to expand radially outwardly to varying degrees along its length to conform to shape of the inner surface 416. In some examples, the docking station 10 is configured such that the sealing portion(s) 410 and/or the retaining portion(s) engage the inner surface 416, even though the shape of the blood vessel or anatomy of the heart vary significantly along the length of the docking station. The docking station can be made from a very resilient or compliant material to accommodate large variations in the anatomy. For example, the docking station 10 can be made from a highly flexible metal, metal alloy, or polymer. Examples of a metals and metal alloys that can be used include, but are not limited to, nitinol, elgiloy, and stainless steel, but other metals and highly resilient or compliant non-metal materials can be used. For example, the docking station 10 can have a frame or portion of a frame (e.g., a self-expanding frame, retaining portion(s), scaling portion(s), valve seat, etc.) made of these materials, e.g., from shape memory materials, such as nitinol. These materials allow the frame to be compressed to a small size, and then when the compression force is released, the frame will self-expand back to its pre-compressed diameter. Docking stations described herein can be self-expanding and/or expandable with an inflatable device to cause the docking station to engage an inner surface 416 having a variable shape.

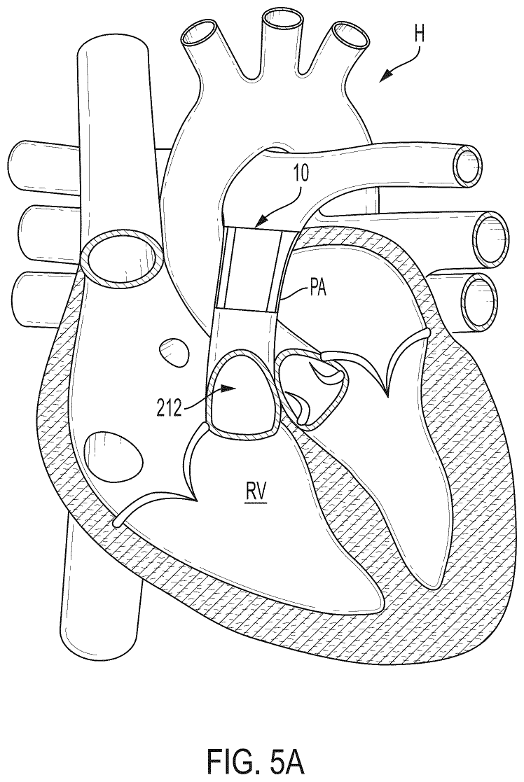

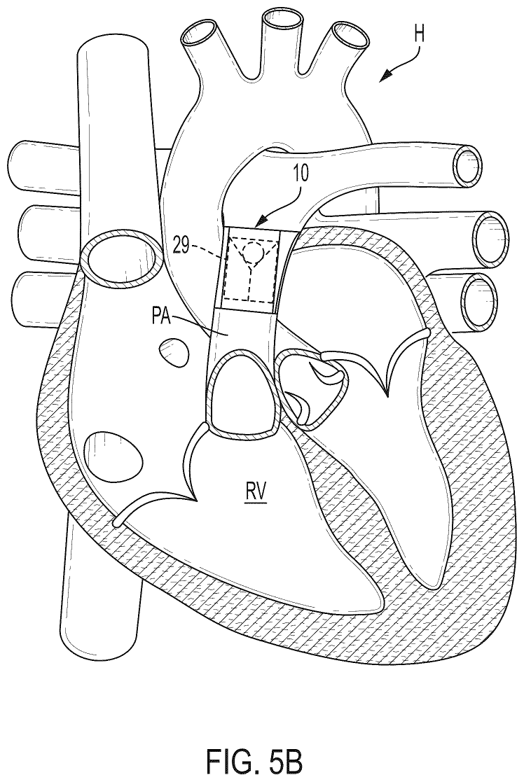

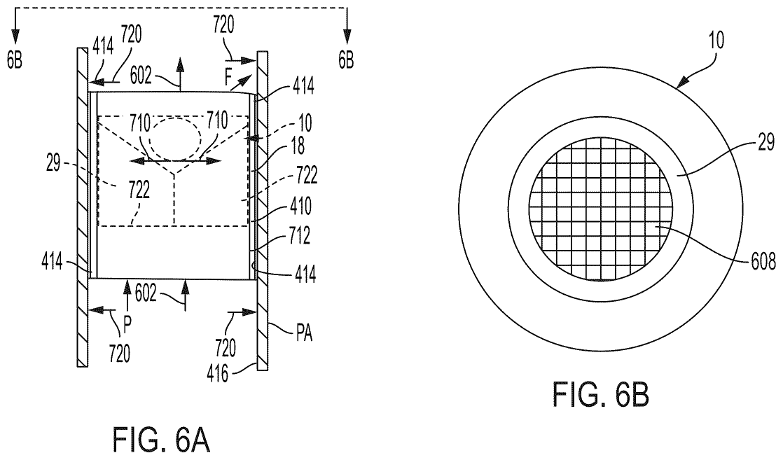

Referring to FIG. 5A, a docking station, e.g., a docking station as described with respect to FIGS. 4A-4D, is deployed in the pulmonary artery PA of a heart H. FIG. 5B illustrates a valve 29 deployed in the docking station 10 illustrated by FIG. 5A. In FIGS. 6A and 6B, the heart is in the systolic phase. FIG. 6A is an enlarged schematic representation of the docking station 10 and valve 29 in the pulmonary artery PA. When the heart is in the systolic phase, the valve 29 opens. Blood flows from the right ventricle RV and through the pulmonary artery PA, docking station 10, and valve 29 as indicated by arrows 602. FIG. 6B illustrates a blood-filled space 608 that represents the valve 29 being open when the heart is in the systolic phase. FIG. 6B does not show the interface between the docking station 10 and the pulmonary artery to simplify the drawing. The cross-hatching in FIG. 6B illustrates blood flow through the open valve. In some examples, blood is prevented from flowing between the pulmonary artery PA and the docking station 10 by the sealing portion(s) 410 and blood is prevented from flowing between the docking station 10 and the valve 29 by seating of the valve 29 in the seat 18 of the docking station 10. In this example, blood is substantially only flowing or only able to flow through the valve 29 when the heart is in the systolic phase.

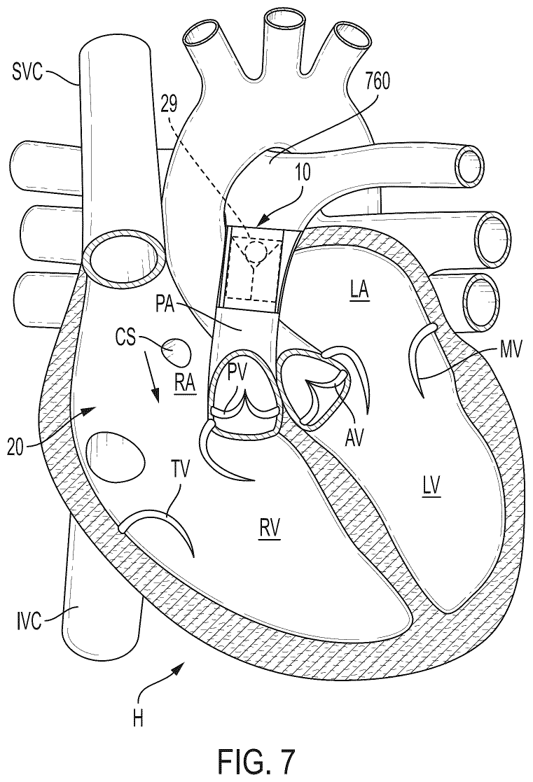

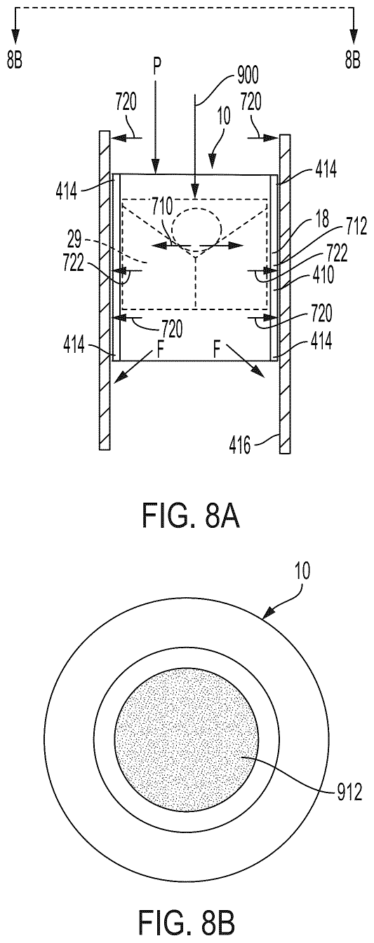

FIG. 7 illustrates the valve 29, docking station 10 and heart H illustrated by FIG. 5B, when the heart is in the diastolic phase. Referring to FIGS. 8A and 8B, when the heart is in the diastolic phase, the valve 29 closes. FIG. 8A is an enlarged schematic representation of the docking station 10 and valve 29 in the pulmonary artery of FIG. 7. Blood flow in the pulmonary artery PA above the valve 29 (i.e., in the pulmonary branch 760) is blocked by the valve 29 being closed and blocking blood flow as indicated by arrow 900. The solid area 912 in FIG. 8B represents the valve 29 being closed when the heart is in the diastolic phase.

In some examples, the docking station 10 acts as an isolator that prevents or substantially prevents radial outward forces of the valve 29 from being transferred to the inner surface 416 of the circulatory system. In some examples, the docking station 10 includes a valve seat 18 (which is not expanded radially outwardly or is not substantially expanded radially outward by the radially outward force of the THV or valve 29, i.e., the diameter of the valve seat is not increased or is increased by less than about 4 mm by the force of the THV), and anchoring/retaining portions 414 and scaling portions 410, which impart only relatively small radially outward forces 720, 722 on the inner surface 416 of the circulatory system (as compared to the radially outward force applied to the valve seat 18 by the valve 29).

When no docking station is used, stents and frames of THVs are held in place in the circulatory system by a relatively high radial outward force 710 of the stent or frame 712 of the THV acting directly on the inside surface 416 of the circulatory system. If a docking station is used, as in the example illustrated by FIG. 6A, the stent or frame 712 of the valve 29 expands radially outward or is expanded radially outward to impart the high force 710 on the valve seat 18 of the docking station 10. This high radially outward force 710 secures the valve 29 to the valve seat 18 of the docking station 10. However, since the valve seat 18 is not expanded or is not substantially expanded by the force 710, the force 710 is isolated from the circulatory system, rather than being used to secure the docking station in the circulatory system.

In some examples, the radially outward force 722 of the sealing portions 410 to the inside surface 416 is substantially smaller than the radially outward force 710 applied by the valve 29 to the valve seat 18. For example, the radially outward sealing force 722 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve. In some examples, the radially outward force 722 of the sealing portions 410 is selected to provide a seal between the inner surface 416 and the sealing portion 410 but is not sufficient by itself to retain the position of the valve 29 and docking station 10 in the circulatory system.

In some examples, the radially outward force 720 of the anchoring/retaining portions 414 to the inside surface 416 is substantially smaller than the radially outward force 710 applied by the valve 29 to the valve seat 18. For example, the radially outward sealing force 720 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve.

In some examples, the radially outward force 720 of the retaining portions 414 is not sufficient by itself to retain the position of the valve 29 and docking station 10 in the circulatory system. Rather, the pressure of the blood 608 is used to enhance the retention of the retaining portions 414 to the inside surface 416. Referring again to FIG. 6A, when the heart is in the systolic phase, the valve 29 is open and blood flows through the valve as indicated by arrows 602. Since the valve 29 is open and blood flows through the valve 29, the pressure P applied to the docking station 10 and valve 29 by the blood is low as indicated by the small arrow P in FIG. 6A. Even though small, the pressure P forces the docking station and its upper retaining portions 414 against the surface 416 generally in the direction indicated by arrow F. This radially outward directed blood flow assisted force F applied by the retaining portions 414 to the surface 416 impedes the docking station 10 and valve 29 from moving in the direction 602 of blood flow in the systolic phase of the heart H.

Referring to FIG. 8A, when the heart is in the diastolic phase, the valve 29 is closed and blood flow is blocked as indicated by arrow 900. Since the valve 29 is closed and the valve 29 and docking station 10 block the flow of blood, the pressure P applied to the docking station 10 and valve 29 by the blood is high as indicated by the large arrow P in FIG. 8A. This large pressure P forces the lower retaining portions 414 against the surface 416 generally in the direction indicated by the large arrows F. This radially outward directed blood flow assisted force F applied by the retaining portions 414 to the surface 416 prevents the docking station 10 and valve 29 from moving in the direction indicated by arrow 900.

Since the force applied by the upper and lower retaining portions 414 is determined by the amount of pressure applied to the valve 29 and docking station 10 by the blood, the force applied to the surface 416 is automatically proportioned. That is, the upper retaining portions are less forcefully pressed against the surface 416 when the heart is in the systolic phase than the lower retaining portions are pressed against the surface 416 when the heart is in the diastolic phase. This is because the pressure against the open valve 29 and docking station 10 in the systolic phase is less than the pressure against the closed valve and docking station in the diastolic phase.

According to an exemplary aspect of the present disclosure, a docking station may include an expandable frame having an outer material (e.g., one or more sheets or tubes of material) attached to an exterior of the frame to define an outer periphery of the docking station.

In some examples, the outer material may promote tissue ingrowth by providing a degree of permeability, such as, for example, a water permeable material. Exemplary materials promoting tissue ingrowth include knitted, woven, or otherwise perforated biocompatible materials. Such material may include, for example, one or more of polyethylene terephthalate (PET), fluorinated ethylene propylene (FEP), polytetrafluoroethylene (PTFE), ethylene tetrafluoroethylene (ETFE), and thermoplastic polyurethane (TPU). A woven/knitted material may be provided with a thread count selected to provide adequate permeability, for example, between about 160 and about 235 threads per inch.

In some examples, the outer material may additionally or alternatively provide additional surface contact and/or surface friction between the docking station and the implantation side, for example, to prevent migration of the docking station (and docked valve or other medical device) within the vessel.

In some examples, the outer material may additionally or alternatively reduce parastent (between the stent and the vessel) or paravalvular (between the stent seat and the implanted medical device) leakage, for example, by filling gaps or discontinuities in the stent contacting surfaces.

In some examples, the outer material may additionally or alternatively provide a layer of cushioning between the docking station frame and the tissue at the implant site, for example, to prevent or reduce erosion or other damage or trauma to the tissue contacted by the frame. For example, an outer material layer (e.g., cloth) may be provided with a thickness selected to provide adequate cushioning (e.g., about 0.05 mm to about 1 mm thick) to limit localized embedding forces of the tissue engaging struts (e.g., at the convex sealing portions of the stent frame).

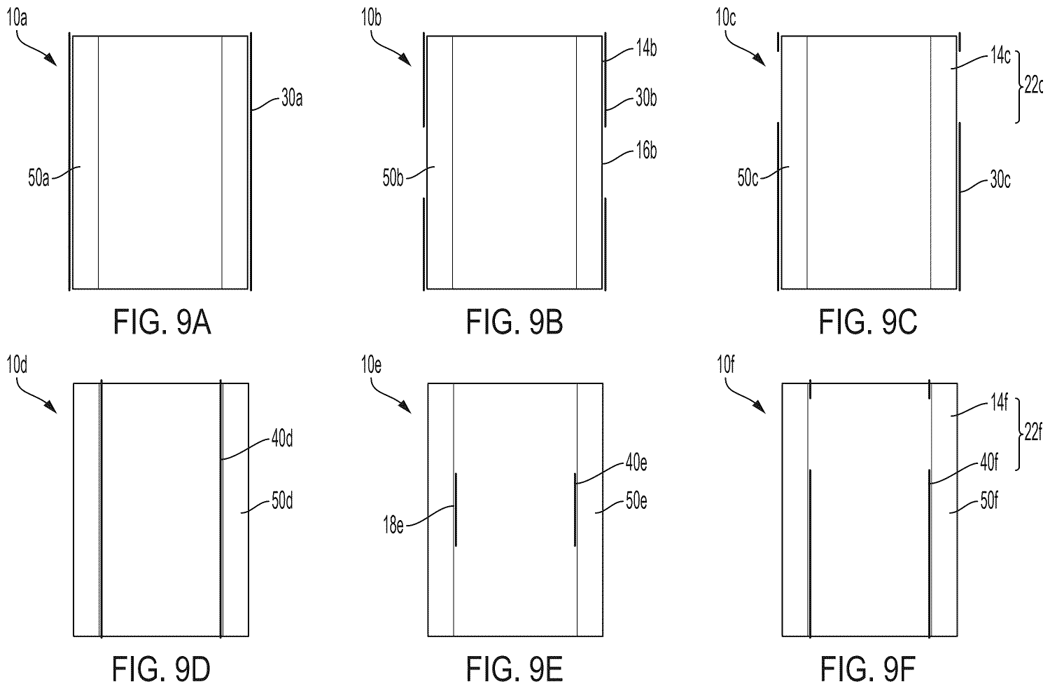

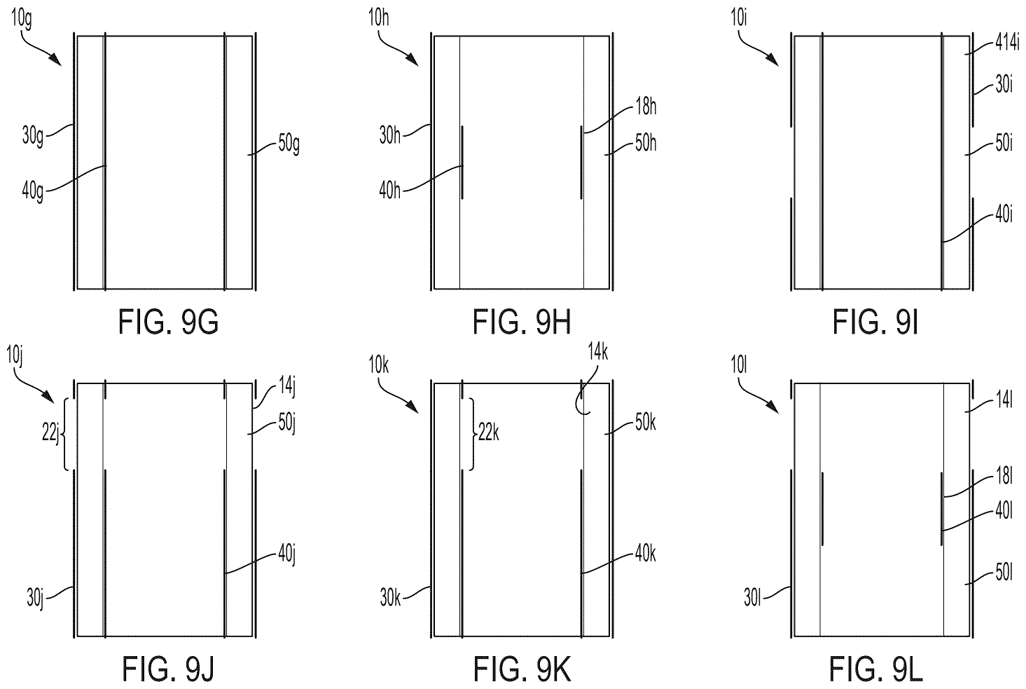

The outer material may be applied to the exterior of the docking station frame in a variety of arrangements and configurations. FIGS. 9A-9C schematically illustrate some such exemplary arrangements and configurations.

In some examples, as shown in FIG. 9A, the docking station 10a includes an outer material 30a that extends to cover the entire exterior surface of the docking station frame 50a.

In some examples, as shown in FIG. 9B, the docking station 10b includes an outer material 30b that may be limited to portions of the frame 50b expected to contact tissue at the implant site, for example, to promote tissue ingrowth, provide additional surface contact and/or surface friction, and/or reduce paravalvular or para-stent leakage. For example, in examples including docking station frames having enlarged or flared end portions and a narrower central waist portion intended to be spaced apart from the implant vessel tissue, the outer material 30b may be limited to these enlarged end portions 14b, with the central waist portion 16b uncovered by the outer material.

In some examples, as shown in FIG. 9C, the docking station 10c includes an outer material 30c that may be positioned so as not to cover at least portions of the outflow end portion 14c of the docking station frame 50c (or other portions of the docking station frame through which blood flow may be desired). This may be accomplished, for example, by truncating the outer material 30c axially inward of the outflow end portion 14c, or by providing one or more openings or cutouts 22c in the outer material in alignment with the outflow end portion (e.g., in alignment with strut defined cells at the outflow end). These openings/cutouts may be formed in the material 30c before or after the material is attached to the docking station frame 50c. In examples where the outer material is provided with openings/cutouts, outer material may be applied to or maintained on the distal end frame portions (e.g., portions bordering open/uncovered cells), for example, to provide increased friction and/or tissue surface contact, endothelialization, and/or protective cushioning at the distal end of the docking station frame 50c.

According to another exemplary aspect of the present disclosure, a docking station may additionally or alternatively include an expandable frame having an inner material (e.g., one or more sheets or tubes of material) attached to an interior of the frame to define an inner periphery of the docking station.

In some examples, the inner material may impede or discourage tissue ingrowth, by providing a degree of impermeability, for example, to minimize interference with the implanted medical device (e.g., valve leaflets) by such tissue ingrowth. Exemplary materials impeding tissue ingrowth include water impermeable materials, films or fabrics that are non-woven or non-perforated (or having smaller perforations), materials that are coated with a biocompatible water impermeable coating. Such material may include, for example, one or more of polyethylene terephthalate (PET), fluorinated ethylene propylene (FEP), polytetrafluoroethylene (PTFE), ethylene tetrafluoroethylene (ETFE), and thermoplastic polyurethane (TPU).

In some examples, the inner material may additionally or alternatively define a seat portion (e.g., valve seat) of the docking station, and may be configured to provide additional surface contact and/or sealing engagement between the docking station and the implanted medical device, for example, to prevent leakage past the outer perimeter of the implanted medical device, and/or to provide more robust retention of the implanted medical device within the docking station.

The outer and inner materials may be applied to the docking station frame in a variety of arrangements and configurations. FIGS. 9D-9F schematically illustrate some such examples.

In some examples, as shown in FIG. 9D, the docking station 10d includes an inner material 40d that extends to cover the entire interior surface of the docking station frame 50d.

In some examples, as shown in FIG. 9E, the docking station 10e includes an inner material 40c that may be limited to portions of the frame 50e expected to contact an implanted medical device, for example, at a seat portion 18e of the docking station.

In some examples, as shown in FIG. 9F, the docking station 10f includes an inner material 40f that may be positioned so as not to cover at least portions of the outflow end portion 14f of the docking station frame 50f (or other portions of the docking station frame through which blood flow may be desired). This may be accomplished, for example, by truncating the inner material 40f axially inward of the outflow end portion 14f, or by providing one or more openings or cutouts 22f in the inner material in alignment with the outflow end portion (e.g., in alignment with strut defined cells at the outflow end). These openings/cutouts may be formed in the material 40f before or after it is attached to the docking station frame 50f.

According to another exemplary aspect of the present disclosure, a docking station may include an expandable frame having an outer material attached to an exterior of the frame and an inner material attached to an interior of the frame, as described above, with at least a portion of the outer material in axial alignment with at least a portion of the inner material.

In some examples, a permeable, tissue ingrowth promoting outer material is at least partially axially aligned with an impermeable, tissue ingrowth impeding inner material, such that the inner material functions as a barrier for endothelial tissue extending through the outer material.

In some examples, axially aligned portions of the outer and inner materials may additionally or alternatively encapsulate or sandwich at least a portion of the docking station frame. In such an arrangement, in the event of fracture in the encapsulated portion of the docking station frame, any loose or broken pieces of the frame are retained by the encapsulating outer and inner materials, and thereby prevented from separating from the docking station and embolizing in or otherwise damaging the implantation vessel.

The outer and inner materials may be applied to the docking station frame in a variety of arrangements and configurations, including, for example, any combination of the outer material configurations of FIGS. 9A-9C and the inner material configurations of FIGS. 9D-9F. FIGS. 9G-9L schematically illustrate some examples.

In some examples, as shown in FIG. 9G, the docking station 10g includes outer and inner materials 30g, 40g that both extend to cover the entire axial length (e.g., the entire exterior and interior surfaces) of the docking station frame 50g, for example, to promote tissue ingrowth, provide additional surface contact and/or surface friction, and/or reduce paravalvular or para-stent leakage, to provide a sealing surface for an implanted medical device, and/or to fully encapsulate the frame struts.

In some examples, as shown in FIG. 9H, the docking station 10h includes an outer material 30h that extends to cover the entire exterior surface of the docking station frame 50h, and an inner material 40h that is substantially limited to the medical device retaining seat portion 18h of the docking station, for example, to provide a sealing surface for an implanted medical device, and/or to prevent tissue ingrowth into the implanted medical device (i.e., with the use of an impermeable inner material).

In some examples, as shown in FIG. 9I, the docking station 10i includes an outer material 30i that is limited to retaining portions 414i of the frame 50i that are expected to contact tissue at the implant site (e.g., axial end portions of the frame), for example, to promote tissue ingrowth, provide additional surface contact and/or surface friction, and/or reduce paravalvular or para-stent leakage, and an inner material 40i that extends to cover the entire interior surface of the docking station frame, for example, to provide scaling engagement of an implanted device at the stent seat portion, and encapsulation of the frame struts at the retaining portions of the frame.

In some examples, as shown in FIG. 9J, the docking station 10j includes outer and inner materials 30j, 40j that are both positioned so as so as not to cover at least portions of the outflow end portion 14j of the docking station frame 50j (as a portion of the docking station frame through which blood flow may be desired), for example, by truncating the outer and inner materials axially inward of the outflow end, or by providing one or more openings or cutouts 22j in the outer and inner materials in alignment with the outflow end. These openings/cutouts may be formed in the materials before or after they are attached to the docking station frame 50j. In examples where the outer material is provided with openings/cutouts, outer material may be applied to the distal end frame portions (e.g., portions bordering open/uncovered cells), for example, to provide increased friction and/or tissue surface contact, endothelialization, or protective cushioning at the distal end of the docking station frame. Likewise, inner material having openings/cutouts may be applied to the distal end frame portions (e.g., portions bordering open/uncovered cells), for example, to join with the outer material at the distal end strut portions to encapsulate and protect the distal end strut portions.

In some examples, as shown in FIG. 9K, the docking station 10k includes an impermeable inner material 40k that is truncated or apertured (as described above) so as so as not to cover at least portions of the outflow end portion 14k of the docking station frame (as a portion of the docking station frame through which blood flow may be desired), while the permeable outer material 30k extends over the entire exterior surface of the docking station frame 50k, providing for increased retaining/sealing tissue engagement and/or tissue ingrowth, while permitting a degree of blood flow through the permeable outer material at the outflow end portion. In examples where the inner material 40k is provided with openings/cutouts 22k, inner material may be applied to the distal end frame portions (e.g., portions bordering open/uncovered cells), for example, to join with the outer material 30k at the distal end strut portions to encapsulate and protect the distal end strut portions.

In some examples, as shown in FIG. 9L, the docking station 101 includes an outer material 301 that is truncated or apertured so as so as not to cover at least portions of the outflow end portion 141 of the docking station frame 501 (as a portion of the docking station frame through which blood flow may be desired), and an inner material 401 that is substantially limited to the medical device retaining seat portion 181 of the docking station. In examples where the outer material is provided with openings/cutouts, outer material may be applied to the distal end frame portions 4141 (e.g., portions bordering open/uncovered cells), for example, to provide increased friction and/or tissue surface contact, endothelialization, or protective cushioning at the distal end of the docking station frame.

In FIGS. 4A through 9L, the docking station frame is schematically illustrated using parallel vertical lines representing an exterior, tissue engaging portion of the frame and an interior, medical device (e.g., valve) receiving portion of the frame. These exterior and interior portions of the frame may take a wide variety of different forms, examples of which are described in greater detail below. In many examples, the exterior and interior portions of the frame may be defined by opposed exterior and interior surfaces of an array of cell-defining struts, such that the exterior and interior surfaces are spaced apart by the thickness of the array of struts, and the outer and inner materials extend over the cells to cover the cell openings defined by or bordered by the struts.

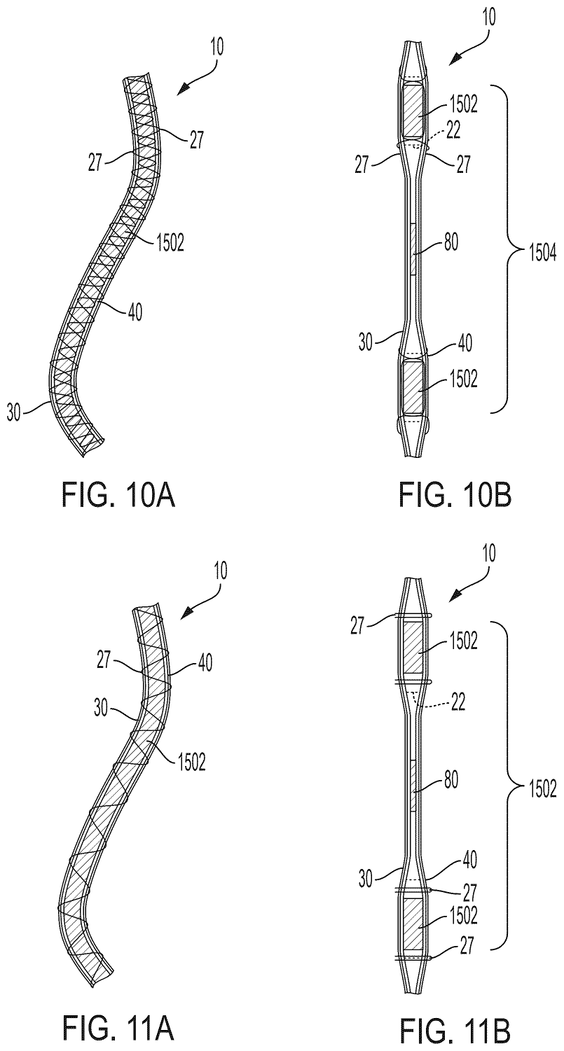

A variety of suitable methods and arrangements may be used to secure the outer and inner materials with the frame. As one example, as schematically illustrated in the partial profile and end cross-sectional views of FIGS. 10A and 10B, the outer and inner materials 30, 40 of a stent or docking station 10 may each be stitched or sutured to the struts, for example, with stitching/suturing thread 27 threaded through the material 30, 40, and laced around the struts. In some examples, as schematically illustrated in the partial profile and end cross-sectional views of FIGS. 11A and 11B, the outer and inner materials 30, 40 may be stitched or sutured to each other, around the outer perimeter and/or within the inner perimeter of the cell 1504, for example, with stitching/suturing thread 27 threaded through the materials 30, 40. Thread/suture material may include, for example, polytetrafluoroethylene (PTFE), high-density polyethylene (HDPE), ultra-high-molecular-weight polyethylene (UHMWPE). In some examples, cither or both of the outer and inner materials 30, 40 may include openings or cutouts 22 within the inner perimeter of one or more of the cells 1504, for example, the cells at the distal end of the stent or docking station 10, to permit or facilitate blood flow through an outflow section of the stent, while still securing or encapsulating the cell-defining struts 1502.

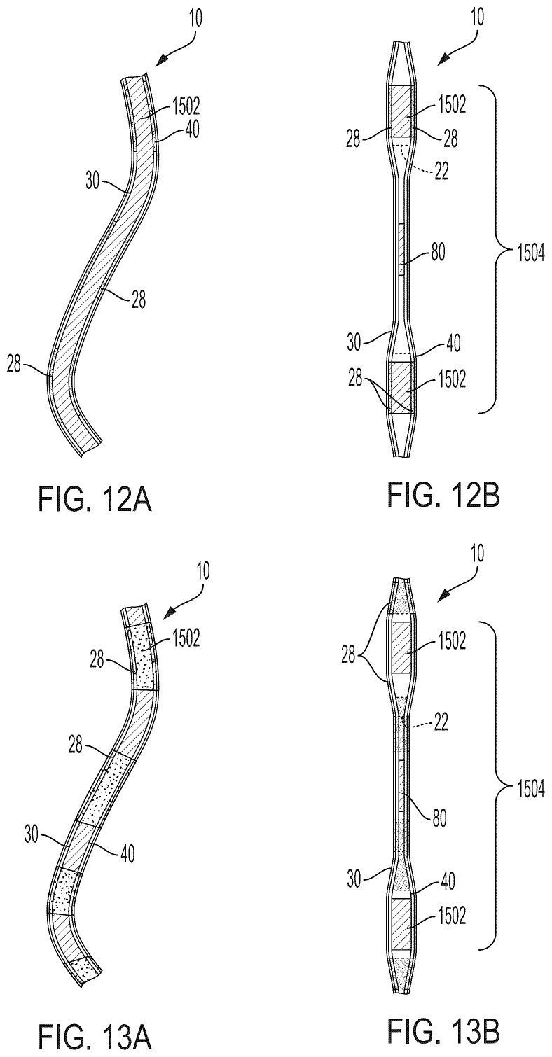

In some examples, as schematically illustrated in the partial profile and end cross-sectional views of FIGS. 12A and 12B, the outer and inner materials 30, 40 may each be adhered to the struts 1502, for example, with one or more layers of adhesive 28 disposed between the inner, frame facing surfaces of the materials and the surfaces of the struts. Such adhesive 28 may be applied to the inner, frame facing surfaces of the materials 30, 40, to the surfaces of the struts 1502, or both, before the materials are brought into contact with the expanded frame 1500. In some examples, as schematically illustrated in the partial profile and end cross-sectional views of FIGS. 13A and 13B, the outer and inner materials 30, 40 may be adhered to each other, for example, with a layer of adhesive 28 disposed between the opposed inner surfaces of the materials, around the outer perimeter and/or within the inner perimeter of the cell 1504. Such adhesive 28 may be applied to the inner frame facing surface of the outer material 30, the inner, frame facing surface of the inner material 40, or both, before the materials are brought into contact with the expanded frame 1500. The adhesive 28 may include a biocompatible adhesive, such as, for example, a moisture curing, light curing, heat curing, or reactive chemistry adhesive. In some examples, either or both of the outer and inner materials 30, 40 may include openings or cutouts 22 within the inner perimeter of one or more of the cells 1504, for example, the cells at the distal end of the stent or docking station 10, to permit or facilitate blood flow through an outflow section of the stent, while still securing or encapsulating the cell-defining struts 1502.

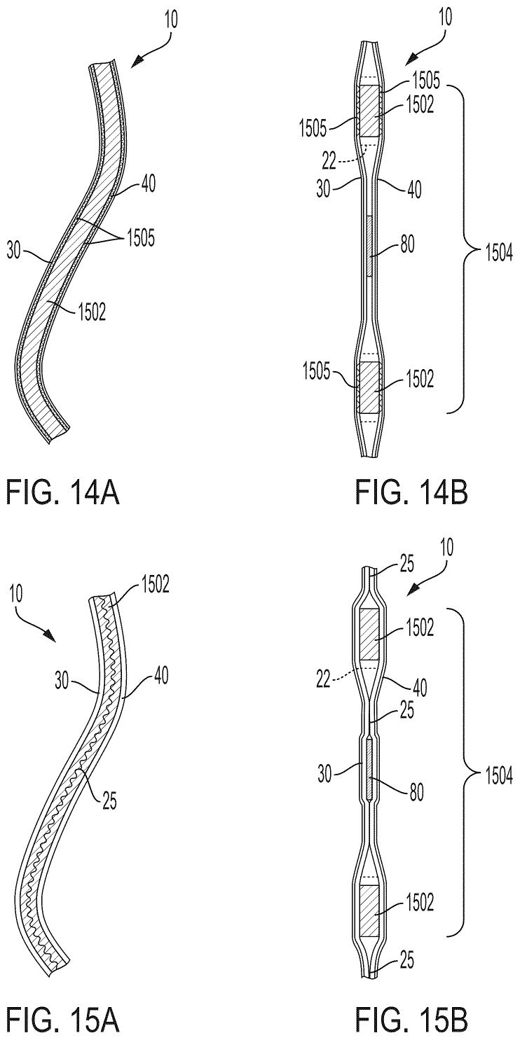

In some examples, as schematically illustrated in the partial profile and end cross-sectional views of FIGS. 14A and 14B, the outer and inner materials 30, 40 may each be heat staked to the struts 1502, for example, with portions of the materials melted/fused to the struts 1502, which may be provided with surface discontinuities 1505 to promote heat staking attachment. In some examples, as schematically illustrated in the partial profile and end cross-sectional views of FIGS. 15A and 15B, the outer and inner materials 30, 40 may be heat fused to each other, for example, at locations 25 around the outer perimeter and/or within the inner perimeter of the cell 1504. In some examples, either or both of the outer and inner materials 30, 40 may include openings or cutouts 22 within the inner perimeter of one or more of the cells 1504, for example, the cells at the distal end of the stent or docking station 10, to permit or facilitate blood flow through an outflow section of the stent, while still securing or encapsulating the cell-defining struts 1502.

The outer and inner materials 30, 40 may be fitted with the expanded frame using a variety of methods and arrangements. In some examples, either or both of the outer and inner materials may be cut from sheets of material in shapes selected to conform with the contours of the expanded frame when the cut material shapes are wrapped around or within the expanded frame and attached along adjoining edges of the cut material shapes. Exemplary patterns of cut material shapes for an expanded stent frame are described in co-owned PCT Application Pub. No. WO 2022/103734, the entire disclosure of which is incorporated herein by reference.

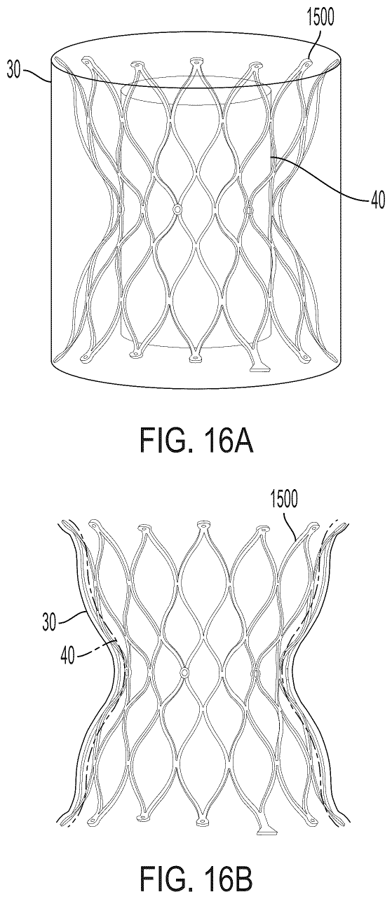

In some exemplary arrangements, either or both of the outer and inner materials may be provided as tubes or sleeves of shrink wrap material that are heat shrunk (for the outer material) into a shape that provides close (e.g., contacting or interference fit) conformance with the expanded frame. Exemplary shrink wrap materials include fluorinated ethylene propylene (FEP), polytetrafluoroethylene (PTFE), ethylene tetrafluoroethylene (ETFE), and thermoplastic polyurethane (TPU). FIG. 16A illustrates an exemplary frame 1500 aligned with tubular shrink wrap materials 30, 40 sized for slip fit concentric alignment with the expanded frame when in a non-thermoformed condition. To provide these materials in a closely conforming condition, the tubular materials may be heat shrunk onto an appropriately sized and shaped mandrel, and then removed from the mandrel for installation over (for the outer material 30) or into (for the inner material 40) the expandable frame 1500. For at least the outer material 30, the tubular material may be heat shrunk directly onto the expanded frame 1500 and cooled/cured in this close conforming (e.g., contacting or interference fit) contoured shape. In some examples, the inner and outer materials may be formed into the desired size and shape using other thermoforming methods. Once shaped to conform with the expanded frame 1500, the materials 30, 40 may be securely attached to the frame, for example, using any one or more of the attachment arrangements shown in FIGS. 10A-15B and described above.

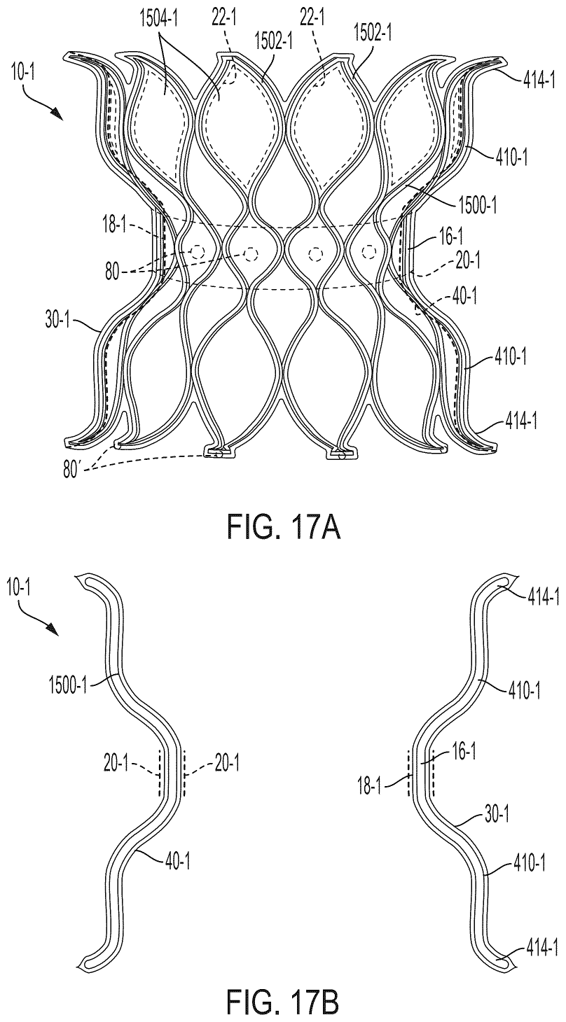

In some examples, a docking station or other such stent may include an hourglass shaped frame, with flared profiled end portions providing flared retaining engagement with the vessel inner surface, a concave profiled central or waist portion defining a valve seat radially spaced from the vessel inner surface, and convex profiled sealing portions between the waist portion and the flared end portions providing tangential scaling engagement with the vessel inner surface. In some examples, as shown in FIGS. 17A and 17B, a docking station or other such stent 10-1 may have an hourglass shaped frame 1500-1 with flared end (retaining) portions 414-1, convex medial (scaling) portions 410-1, and a concave central waist portion 16-1. Similar frame configurations are shown and described in greater detail in co-owned U.S. Pat. No. 10,363,130, the entire disclosure of which is incorporated herein by reference. In some examples, as shown in FIGS. 17A and 17B, an outer material 30-1 (e.g., any of the exemplary materials described above) is secured to the exterior of the frame 1500-1 (e.g., using any of the methods or arrangements described above), and an inner material 40-1 (e.g., any of the exemplary materials described above) is secured to the interior of the frame (e.g., using any of the methods or arrangements described above). In some examples, either or both of the outer and inner materials 30-1, 40-1 may include openings or cutouts 22-1 within the inner perimeter of one or more of the cells 1504-1, for example, the cells at the distal end of the stent or docking station 10-1, to permit or facilitate blood flow through an outflow section of the stent, while still securing or encapsulating the cell-defining struts 1502-1. In some examples, as shown, either or both of the outer and inner materials 30-1, 40-1 extend over and/or beyond the frame apices at either or both ends of the frame, for example, to provide additional tissue protecting cushioning for these edge portions.

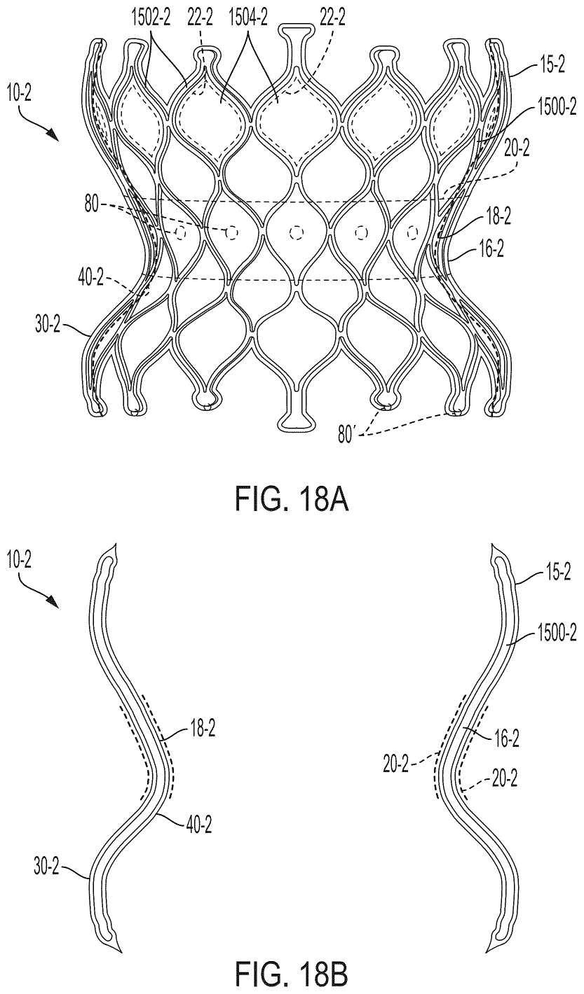

In some examples, a docking station or other such stent may include an hourglass shaped frame, with convex profiled end portions providing tangential retaining and scaling engagement with the vessel inner surface, and a concave profiled central or waist portion defining a valve seat radially spaced from the vessel inner surface. In some examples, as shown in FIGS. 18A and 18B, a docking station or other such stent 10-2 may have an exemplary hourglass shaped frame 1500-2 with convex end portions 15-2 and a concave waist portion 16-2. Similar frame configurations are shown and described in greater detail in co-owned PCT Application Pub. No. WO 2022/103734, the entire disclosure of which is incorporated herein by reference. In some examples, as shown in FIGS. 18A and 18B, an outer material 30-2 (e.g., any of the exemplary materials described above) is secured to the exterior of the frame 1500-2 (e.g., using any of the methods or arrangements described above), and an inner material 40-2 (e.g., any of the exemplary materials described above) is secured to the interior of the frame (e.g., using any of the methods or arrangements described above). In some examples, either or both of the outer and inner materials 30-2, 40-2 may include openings or cutouts 22-2 within the inner perimeter of one or more of the cells 1504-2, for example, the cells at the distal end of the stent or docking station 10-2, to permit or facilitate blood flow through an outflow section of the stent, while still securing or encapsulating the cell-defining struts 1502-2. In some examples, as shown, either or both of the outer and inner materials 30-2, 40-2 extend over and/or beyond the frame apices at either or both ends of the frame, for example, to provide additional tissue protecting cushioning for these edge portions.

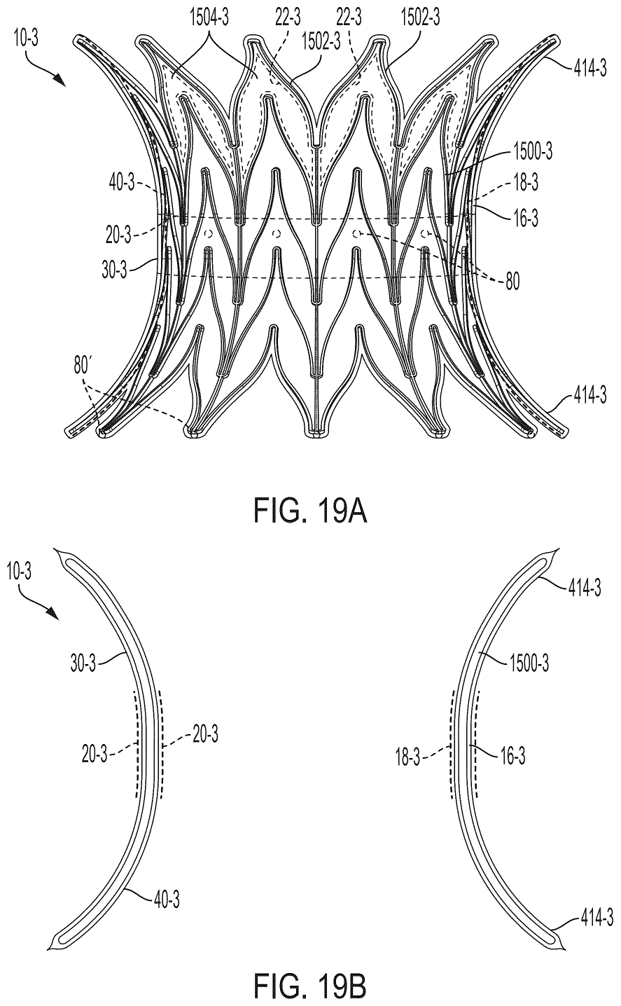

In some examples, a docking station or other such stent may include a frame having a concave profile extending from flared retaining/scaling end portions to a narrower cylindrical or shallow concave waist portion defining a valve seat that may, but need not, be radially spaced from the vessel inner surface. FIGS. 19A and 19B illustrate some examples of docking stations or other such stents 10-3 having a frame 1500-3 including a concave profile extending from flared retaining/scaling end portions 414-3 to a narrower cylindrical or shallow concave waist portion 16-3 defining a seat portion 18-3. Similar frame configurations are shown and described in greater detail in co-owned PCT Application Serial No. PCT/US2022/043296, filed Sep. 13, 2022, the entire disclosure of which is incorporated herein by reference. In some examples, as shown in FIGS. 19A and 19B, an outer material 30-3 (e.g., any of the exemplary materials described above) is secured to the exterior of the frame 1500-3 (e.g., using any of the methods or arrangements described above), and an inner material 40-3 (e.g., any of the exemplary materials described above) is secured to the interior of the frame (e.g., using any of the methods or arrangements described above). In some examples, either or both of the outer and inner materials 30-3, 40-3 may include openings or cutouts 22-3 within the inner perimeter of one or more of the cells 1504-3, for example, the cells at the distal end of the stent or docking station 10-3, to permit or facilitate blood flow through an outflow section of the stent, while still securing or encapsulating the cell-defining struts 1502-3. In some examples, as shown, either or both of the outer and inner materials 30-3, 40-3 extend over and/or beyond the frame apices at either or both ends of the frame, for example, to provide additional tissue protecting cushioning for these edge portions.

In some examples, as shown in FIGS. 17A-19B, the expandable stent or docking station 10-1, 10-2, 10-3 may include a band 20-1, 20-2, 20-3 that extends about the waist or narrow portion 16-1, 16-2, 16-3, or is integral to the waist to form an unexpandable or substantially unexpandable seat portion (e.g., valve seat) 18-1, 18-2, 18-3. The band 20-1, 20-2, 20-3 stiffens the waist and, once the docking station is deployed and expanded, makes the waist/seat relatively unexpandable in its deployed configuration. The unexpandable or substantially unexpandable valve seat 18-1, 18-2, 18-3 prevents the radially outward force of the valve (or other implanted device) from being transferred to the inside surface of the circulatory system. However, in some examples, the waist/valve seat of the deployed docking station can optionally expand slightly in an elastic fashion when the valve is deployed against it. This optional elastic expansion of the waist may put pressure on the valve to help hold the valve in place within the docking station. According to an aspect of the present disclosure, the band 20-1, 20-2, 20-3 may comprise a suture, ring, or strip of material secured to at least one of the outer material 30-1, 30-2, 30-3 and the inner material 40-1, 40-2, 40-3, for example, by sewing, suturing, adhesive, or some other arrangement. Where the band 20-1, 20-2, 20-3 is secured to the inner material 40-1, 40-2, 40-3, the band may also form a valve seat defining surface for the implanted device.

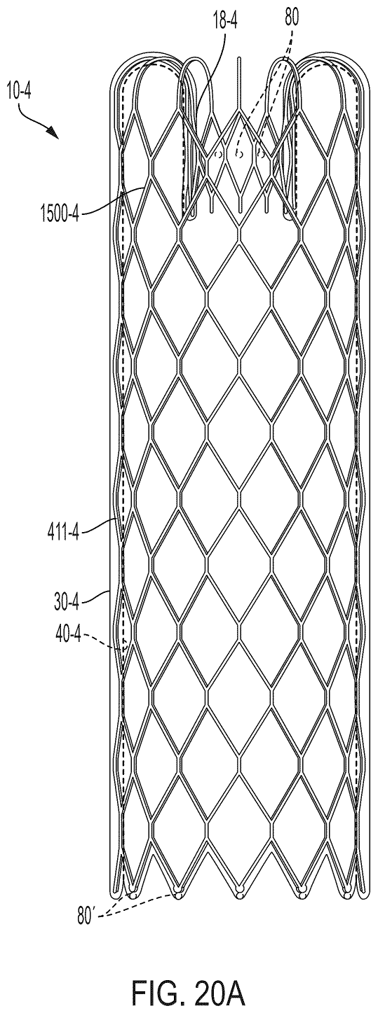

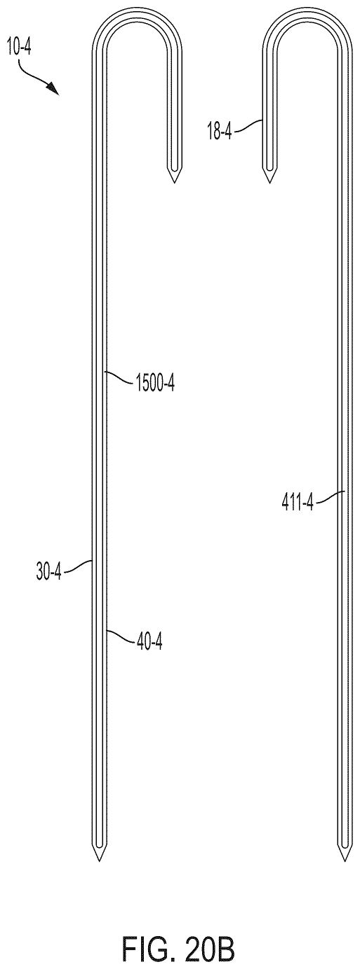

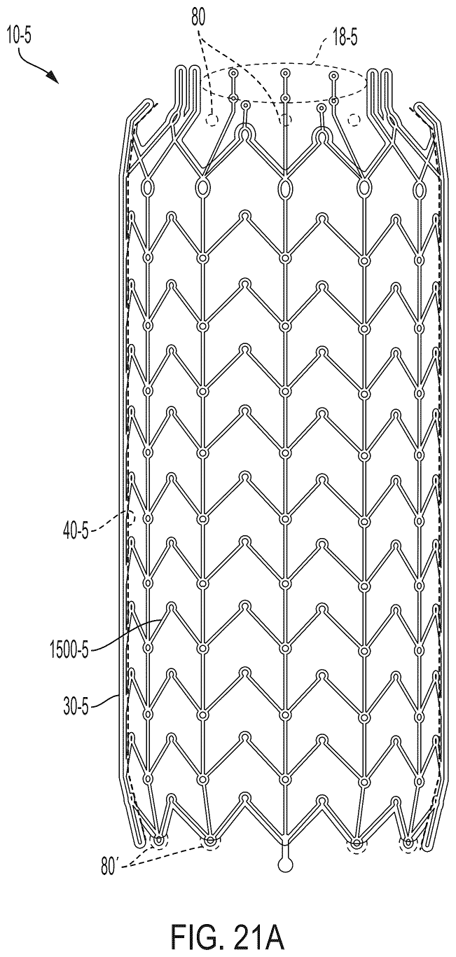

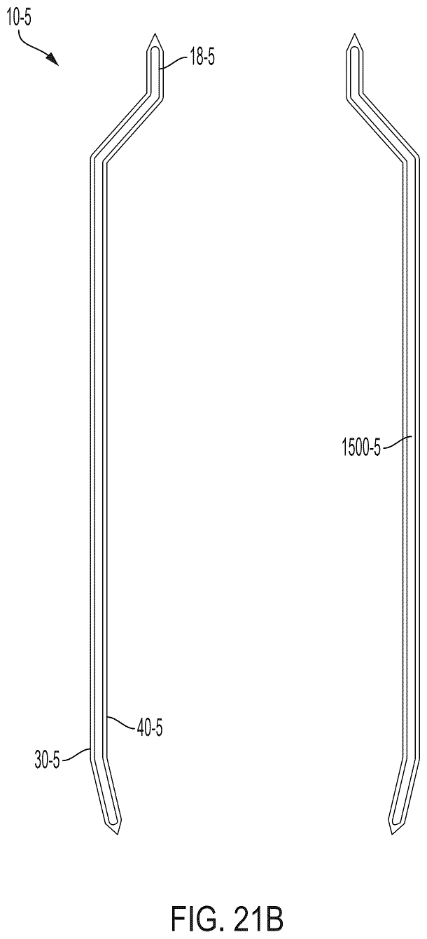

In some examples, a docking station or other such stent may include an elongated cylindrical shaped docking station frame providing a substantially uniform retaining and sealing profile in the expanded, unconstrained state, with radially inward extending legs supporting a valve seat radially inward of the cylindrical retaining/scaling profile portions. FIGS. 20A-20B and 21A-21B illustrate some examples of docking stations or other such stents or docking stations 10-4, 10-5 having exemplary frames 1500-4, 1500-5 including a cylindrical shaped outer frame portion 411-4, 411-5 with a radially inward offset valve seat portion 18-4, 18-5 disposed at a distal or second end of the frame 1500-4, 1500-5. Similar frame configurations are shown and described in greater detail in co-owned PCT Application Pub. No. WO 2022/040120, the entire disclosure of which is incorporated herein by reference. In some examples, an outer material 30-4, 30-5 (e.g., any of the exemplary materials described above) is secured to the exterior of the frame 1500-4, 1500-5 (e.g., using any of the methods or arrangements described above), and an inner material 40-4, 40-5 (e.g., any of the exemplary materials described above) is secured to the interior of the frame (e.g., using any of the methods or arrangements described above). In some examples, as shown, either or both of the outer and inner materials 30-4, 30-5, 40-4, 40-5 extend over and/or beyond the frame apices at either or both ends of the frame, for example, to provide additional tissue protecting cushioning for these edge portions.

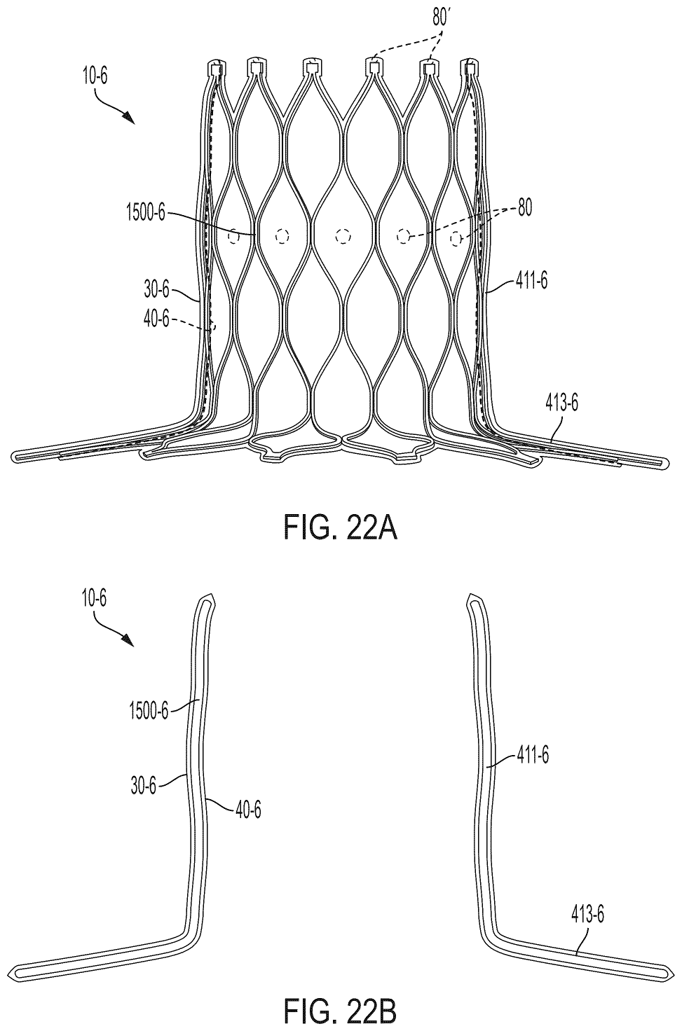

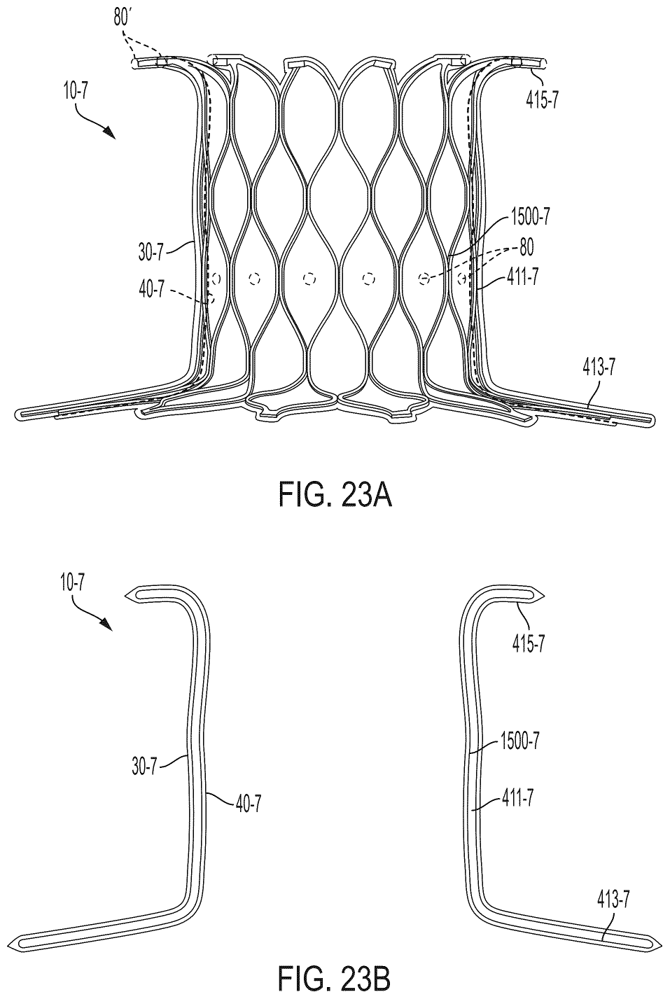

In some examples, a docking station or other such stent may include an elongated cylindrical shaped docking station frame providing a substantially uniform valve seat defining portion in the expanded, unconstrained state, with radially outward extending flexible flanged portions defining retaining and scaling portions at one or both ends of the frame. FIGS. 22A-22B and 23A-23B illustrate some examples of docking stations or other such stents 10-6, 10-7 having exemplary frames 1500-6, 1500-7 including an elongated cylindrical portion 411-6, 411-7 and radially outward extending flexible flanged portions 413-6, 413-7, 415-7 at one (FIGS. 22A-22B) or both (FIGS. 23A-23B) ends of the frame. An outer material 30-6, 30-7 (e.g., any of the exemplary materials described above) is secured to the exterior of the frame 1500-6, 1500-7 (e.g., using any of the methods or arrangements described above), and an inner material 40-6, 40-7 (e.g., any of the exemplary materials described above) is secured to the interior of the frame (e.g., using any of the methods or arrangements described above). In some examples, as shown, either or both of the outer and inner materials 30-6, 40-6, 30-7, 40-7 extend over and/or beyond the frame apices at either or both ends of the frame, for example, to provide additional tissue protecting cushioning for these edge portions. Similar frame configurations are shown and described in greater detail in co-owned PCT Application Pub. No. WO 2022/103734, the entire disclosure of which is incorporated herein by reference.

As shown in FIGS. 17A-23B, the outer and inner materials may extend over the entire length of the frame, for example, as shown in FIG. 9G. In such an arrangement, the extension of the outer material over the tissue engaging sealing portions and retaining portions may promote tissue ingrowth, provide increased friction and/or tissue surface contact for impeding docking station slippage/migration, reduce paravalvular or parastent leakage, and/or provide a protective cushioning layer between the metal frame and the engaged tissue. The extension of the inner material over the seat portion may impede tissue ingrowth, provide a seat portion facilitating retention of an installed device (e.g., prosthetic valve), and/or provide a leak tight seal between the installed device and the frame. The axial alignment of the outer and inner materials may provide for encapsulation of the interposed or sandwiched portions of the frame, for example, to retain loose or broken pieces of the frame, preventing such pieces from separating from the docking station and embolizing in or otherwise damaging the implantation vessel.

According to another exemplary aspect of the present disclosure, the encapsulating outer and inner materials of an expandable stent may be used to retain and/or protect one or more radiopaque markers which can assist with deployment of the expandable stent as well as placement of an implantable medical device into a seat portion of the expandable stent. The one or more radiopaque markers can be radiopaque or have a higher radiopacity such that the one or more radiopaque markers can be identified under fluoroscopy or a similar imaging process. The one or more radiopaque markers can comprise any material or combination of materials that are radiopaque or increase the radiopacity of at least a portion of the expandable stent (e.g., a seat portion or an endmost portion). For example, the one or more radiopaque markers can comprise barium sulfate, bismuth, tungsten, tantalum, platinum-iridium, gold, or any other material which is opaque to fluoroscopy, X-rays, or similar radiation. The radiopaque markers may be disc-shaped and circular or octagonal. However, the one or more radiopaque markers can be configured to reduce axial motion and can be any suitable shape. For example, the one or more radiopaque markers can be hexagonal, triangular, rectangular, elliptical, 3D, or any other shape or configuration. The radiopaque markers can also include an aperture extending through a central portion of the marker. The aperture can be sized such that a suture can pass therethrough. Exemplary radiopaque markers are shown and described in co-owned PCT Application Pub. No. WO 2022/103734 and PCT Application Serial No. PCT/US2022/043296, the entire disclosures of which are incorporated herein by reference.

In some examples, as shown in FIGS. 10B-15B, a radiopaque marker 80 may be encapsulated or captured between the outer and inner materials 30, 40 on the frame 1500, within the inner perimeter of one or more of the cells 1504. Where the materials 30, 40 are affixed to the frame struts 1502 around the cell perimeter (e.g., by suturing/stitching, adhesive, or heat fixation), the radiopaque marker 80 may be loosely retained between the materials within the cell 1504. In some examples, the radiopaque marker 80 may be secured directly to either or both of the outer and inner materials 30, 40, for example, by suturing/stitching, adhesive, or heat fixation of the material(s) 30, 40 to the radiopaque marker 80.

One or more radiopaque markers may be encapsulated by the outer and inner materials at a variety of locations on the frame. For example, as shown in FIGS. 17A-23A, radiopaque markers 80 may be encapsulated between the materials within cells aligned with a seat portion 18-1, 18-2, 18-3, 18-4, 18-5, 18-6, 18-7 of the frame 1500-1, 1500-2, 1500-3, 1500-4, 1500-5, 1500-6, 1500-7, for example, to facilitate location (e.g., under fluoroscopy or a similar imaging process) of the seat portion during implantation of a prosthetic valve or other implantable medical device. Arrangements for providing and using radiopaque markers at a seat portion of a docking station are described in the above-incorporated PCT Application Pub. No. WO 2022/103734.

In some examples, as shown in FIGS. 17A-23A, radiopaque markers 80′ may be encapsulated between the materials at or beyond the apices of the endmost cells, at either or both ends of the frame, for example, to facilitate location (e.g., under fluoroscopy or a similar imaging process) of these frame apices during implantation of a prosthetic valve or other implantable medical device, for example, to avoid impact between the device/valve installing catheter and one or more inward deflected apices. Arrangements for providing and using radiopaque markers at apices of a docking station are described in the above-incorporated PCT Application Serial No. PCT/US2022/043296, filed Sep. 13, 2022.

As discussed above, an expandable docking station may be provided with an outer material configured to provide a layer of cushioning between the docking station frame and the tissue at the implant site, for example, to prevent or reduce erosion or other damage or trauma to the tissue contacted by the frame. In some examples, a fabric (e.g., high density polyethylene terephthalate or PET) skirt or cover is attached (e.g., sutured) on the exterior or outer diameter surface of the docking station frame, for example to provide a thin cushion between the frame and the tissue at the implant site. For example, an outer material layer may be provided with a thickness (e.g., about 0.05 mm to about 0.07 mm thick) sufficient to provide adequate cushioning to limit localized embedding forces of the tissue engaging struts (e.g., at the convex sealing portions of the stent frame), while limited (sufficiently thin) such that the compressed or unexpanded docking station may maintain a diameter (or crimp profile) that can still be accommodated by the standard catheter tube used to deliver the docking station.

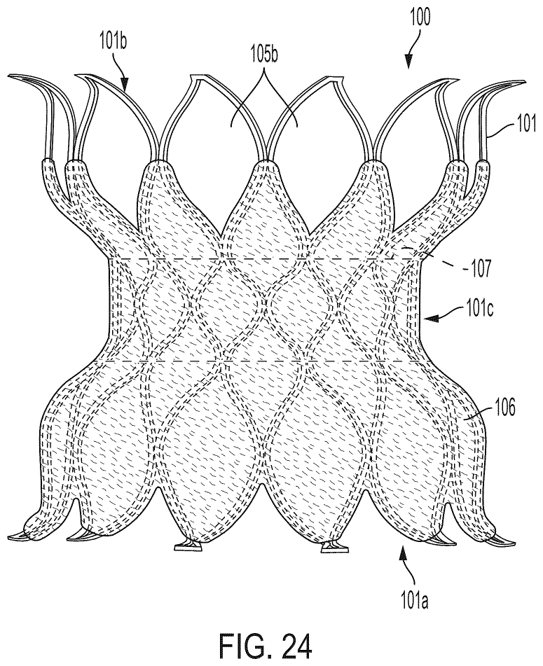

In some examples, as shown in FIG. 24, an expandable stent or docking station 100 includes an exterior cover or skirt 106 covering at least a tissue engaging portion of a docking station frame 101, to provide a layer of cushioning between the docking station frame and the tissue at the implant site. In the illustrated examples, the docking station frame 101 is similar to the docking station frame 1500-1 of FIGS. 17A and 17B. In some examples, a similar cover or skirt configuration may be applied to any suitable docking station frame, including, for example, the docking station frames shown in FIGS. 18A-23B and described above.

The skirt or exterior cover 106 may be provided in a permeable or semi-permeable material selected to promote healing and ingrowth of the implant site tissue, such as, for example, polyethylene terephthalate (PET), low density polyethylene (LDPE), high density polyethylene (HDPE), ultra-high molecular weight polyethylene (UHMWPE), and expanded polytetrafluoroethylene (ePTFE). In some examples, the material may be woven, for example, to further enhance material strength and/or permeability. Exemplary woven materials may be formed, for example, from yarns having a linear density between about 20 decitex and about 25 decitex, or a thread density between about 160 picks per inch and about 225 picks per inch.

In some examples, a selected material may be configured to degrade over time (e.g., about 6 months to about 18 months), with the cushioning function of the material being replaced by that of endothelialized tissue growth at the implant site. In some such examples, the skirt may be provided in biodegradable or bioresorbable polymeric materials such as, for example, poly-1-lactic acid (PLLA), polycaprolactone (PCL), and poly(4-hydroxybutyrate) (P4HB), or one or more biodegradable or bioresorbable polymeric materials blended with a polyethylene (e.g., PET) or other such polymer.

The docking station frame can have an inflow end portion 101a and an outflow end portion 101b. In the illustrated example, the exterior cover 106 is positioned so as not to cover at least portions of the outflow end portion 101b of the docking station frame 101 (e.g., to permit or facilitate blood flow through an outflow section of the docking station), with the cover material being truncated axially inward of the outflow end portion, along the axially inner horizontal strut(s) of the distal row of cells 105b of the docking station frame. In some examples, the exterior cover material may be truncated at a different location along the length of the cover material (e.g., around an axial midpoint of the distal row of cells of the docking station frame) or may extend over the entire axial length of the docking station frame (e.g., with openings or cutouts aligned with the distal row of cells, as described above and schematically shown in FIG. 9C).

In the illustrated example, the docking station 100 includes an inner material 107 around a waist portion 101c of the frame 101 for seating and/or sealing contact with an implanted medical device (e.g., prosthetic valve). This inner material 107 may be limited to the waist portion 101c (e.g., covering a middle row of cells of the docking station frame) of the frame 101, as shown (e.g., as schematically shown in FIG. 9E and described above), or may cover additional portions of the frame. The inner material 107 may be provided in any suitable material (e.g., PET) and may have a thickness (e.g., between about 0.1 mm and about 0.15 mm) sufficient for sealing retention of a catheter installed and expanded prosthetic valve or other such implanted medical device (as shown and described herein), and may be substantially impermeable, for example, to inhibit tissue ingrowth into the inner diameter of the docking station frame 101.

In some examples, an expandable docking station may be provided with a thicker outer material (e.g., between about 0.2 mm and about 0.6 mm thick, or about 0.4 mm thick) configured to provide a thicker layer of cushioning between the docking station frame and the tissue at the implant site, for example, to prevent or reduce erosion or other damage or trauma to the tissue contacted by the frame. In some examples, a compressible fabric (e.g., high density polyethylene terephthalate or PET) skirt or cover is attached (e.g., sutured) on the exterior or outer diameter surface of the docking station frame, for example to provide a thicker, compressible cushion between the frame and the tissue at the implant site.

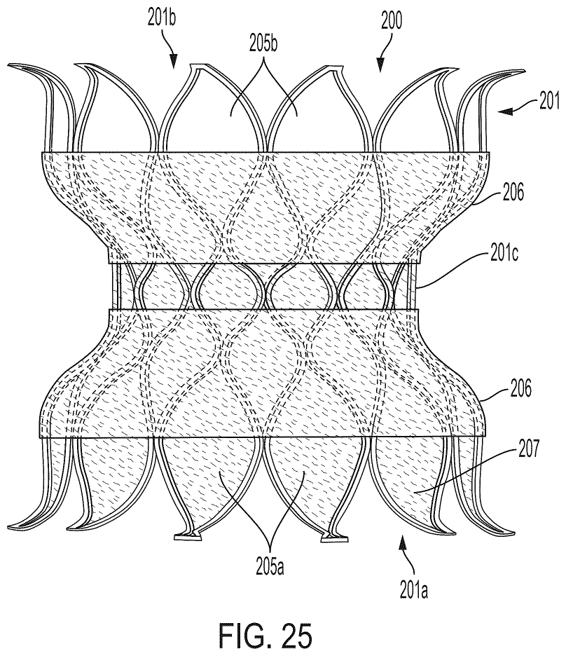

In some examples, as shown in FIG. 25, an expandable stent or docking station 200 includes an exterior cover or skirt 206 covering at least a tissue engaging portion of a docking station frame 201, to provide a layer of cushioning between the docking station frame and the tissue at the implant site. As shown, the docking station frame 201 may be similar to the docking station frame 1500-1 of FIGS. 17A and 17B. In some examples, a similar cover or skirt configuration may be applied to any suitable docking station frame, including, for example, the docking station frames shown in FIGS. 18A-23B and described above.

The exterior cover 206 may be provided in a compressible material selected to provide a desired degree of compressibility (e.g., up to about 15% to about 20% reduction in thickness under compression in a deployed condition of the expandable stent), for example, a crochet knit material having a looped/open structure. In some examples, the material may be highly permeable to water or saline while providing suitable scaling to biological fluids (e.g., platelets, fibrin, red blood cells). The material may additionally or alternatively have a stretchability between about 40% and about 60%, which may vary, for example, based on yarn type and/or density. In some examples, other relatively thick compressible materials may be utilized, such as, for example, polymer foam (e.g., formed by 3D printed, lyophilization, or salt leaching), and nonwoven textile materials (e.g., electrospun, melt spun).

In the illustrated example, the exterior cover is positioned so as not to cover at least portions of the inflow and outflow end portions 201a, 201b and/or the waist portion 201c of the docking station frame 201 (e.g., to limit cushioning to portions having substantial scaling contact with the implant site), with the cover material being truncated axially inward of the outflow and inflow end portions, around an axial midpoint of the distal and proximal rows of cells 205a, 205b of the docking station. In some examples, the exterior cover material may be truncated at a different location along the length of the cover material 206 (e.g., along the axially inner horizontal strut(s) of the proximal and/or distal rows of cells 205a, 205b of the docking station frame), or may extend over the entire axial length of the docking station frame 201.

In the illustrated example, the docking station 200 includes an inner material 207 extending from a proximal end of the docking station frame 201 to the axially inner horizontal strut(s) of the distal row of cells 205b, for example, for seating and/or scaling contact with an implanted medical device (e.g., prosthetic valve) and to provide additional scaling against the pressurized blood at the inflow end of the docking station. The inner material 207 may be provided in any suitable material (e.g., PET) and may have a thickness (e.g., between about 0.1 mm and about 0.15 mm) sufficient for sealing retention of a catheter installed and expanded prosthetic valve or other such implanted medical device, and may be substantially impermeable, for example, to inhibit tissue ingrowth into the inner diameter of the docking station frame 201.

In some examples, an expandable docking station may be provided with a thicker (e.g., between about 0.4 mm and about 0.6 mm thick), bidirectionally stretchable outer material configured to provide a thicker layer of cushioning between the docking station frame and the tissue at the implant site (e.g., to prevent or reduce erosion or other damage or trauma to the tissue contacted by the frame), while being stretchable in both X (circumferential) and Y (axial) directions, for example, to permit both axial stretching of the material in the compressed or unexpanded condition of the frame, and circumferential stretching of the material in the deployed or expanded condition of the frame. In some examples, a bidirectionally stretchable fabric (e.g., a honeycomb cloth) skirt or cover is attached (e.g., sutured) on the exterior or outer diameter surface of the docking station frame, for example to provide a thicker, bidirectionally stretchable compressible cushion between the frame and the tissue at the implant site. The axial stretchability of the outer material allows the compressed or unexpanded docking station to maintain a diameter (or crimp profile) that can still be accommodated by the standard catheter tube used to deliver the docking station, while the circumferential stretchability of the outer material facilitates expansion of the docking station frame upon deployment.

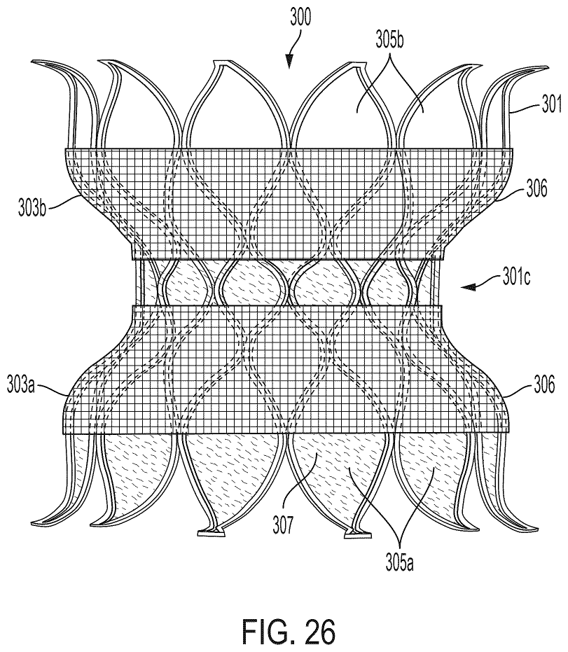

In some examples, as shown in FIG. 26, an expandable stent or docking station 300 may include an exterior cover or skirt 306 covering at least a tissue engaging portion of a docking station frame 301, to provide a bidirectionally stretchable layer of cushioning between the docking station frame and the tissue at the implant site. As shown, the docking station frame 301 may be similar to the docking station frame 1500-1 of FIGS. 17A and 17B. In some examples, a similar cover or skirt configuration may be applied to any suitable docking station frame, including, for example, the docking station frames shown in FIGS. 18A-23B and described above.

The exterior cover 306 may be provided in a bidirectionally stretchable material selected to provide a desired degree of stretchability (e.g., between about 60% and about 100% stretchability in the X and Y directions), for example, a honeycomb cloth polyethylene terephthalate (PET) material. In some examples, the material may be highly permeable to water or saline while providing suitable sealing to biological fluids (e.g., platelets, fibrin, red blood cells).

In the illustrated example, the exterior cover 306 is positioned so as to only cover the convex sealing portions 303a, 303b of the inflow and outflow end portions of the docking station frame 301 (e.g., to limit cushioning to portions having substantial sealing contact with the implant site), with the cover material being omitted from the central waist portion 301c of the docking station frame, for example, to allow the waist portion to be sufficiently expanded during medical device (e.g., prosthetic valve) installation, or to be over-expanded to permit prosthetic valve installation within the inner diameter of a failed prosthetic valve (i.e., a “valve-in-valve” procedure). In some examples, the exterior cover material 306 may be truncated at a different location along the length of the cover material (e.g., along the axially inner horizontal strut(s) of the proximal and/or distal rows of cells 305a, 305b of the docking station frame 301), or may extend over the entire axial length of the docking station frame.

In the illustrated example, the docking station 300 includes an inner material 307 extending from a proximal end of the docking station frame 301 to the axially inner horizontal strut(s) of the distal row of cells 305b, for example, for seating and/or sealing contact with an implanted medical device (e.g., prosthetic valve) and to provide additional sealing against the pressurized blood at the inflow end of the docking station 300. The inner material 307 may be provided in any suitable material (e.g., PET) and may have a thickness (e.g., between about 0.1 mm and about 0.15 mm) sufficient for sealing retention of a catheter installed and expanded prosthetic valve or other such implanted medical device, and may be substantially impermeable, for example, to inhibit tissue ingrowth into the inner diameter of the docking station frame 301.

In some examples, an expandable docking station may be provided with a substantially impermeable outer material (e.g., thermoplastic polyurethane or TPU) configured to provide a layer of cushioning between the docking station frame and the tissue at the implant site (e.g., to prevent or reduce erosion or other damage or trauma to the tissue contacted by the frame), while inhibiting tissue ingrowth, for example to minimize thrombus and/or to maximize perfusion through the open, uncovered portions of the outflow end of the docking station frame. In some examples, a substantially impermeable skirt or cover may be attached (e.g., wrapped) on the exterior or outer diameter surface of the docking station frame, for example to provide a thin cushion between the frame and the tissue at the implant site. For example, an outer material layer may be provided with a thickness (e.g., about 0.07 mm to about 0.20 mm thick) sufficient to provide adequate cushioning to limit localized embedding forces of the tissue engaging struts (e.g., at the convex sealing portions of the stent frame), while limited (sufficiently thin) such that the compressed or unexpanded docking station may maintain a diameter (or crimp profile) that can still be accommodated by the standard catheter tube used to deliver the docking station.

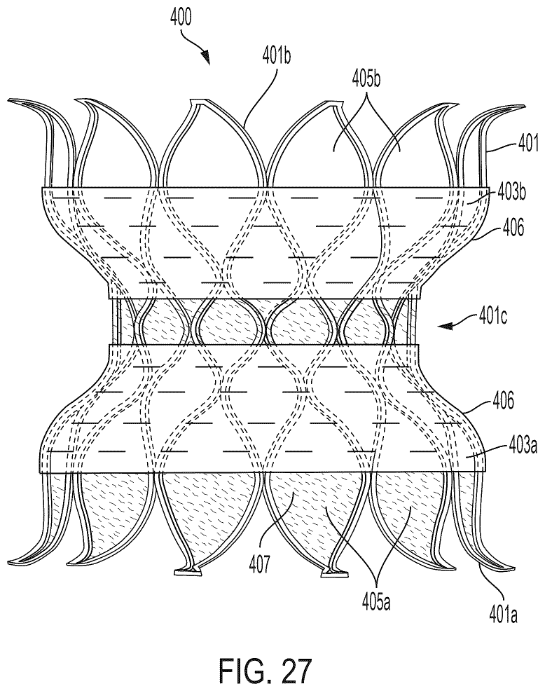

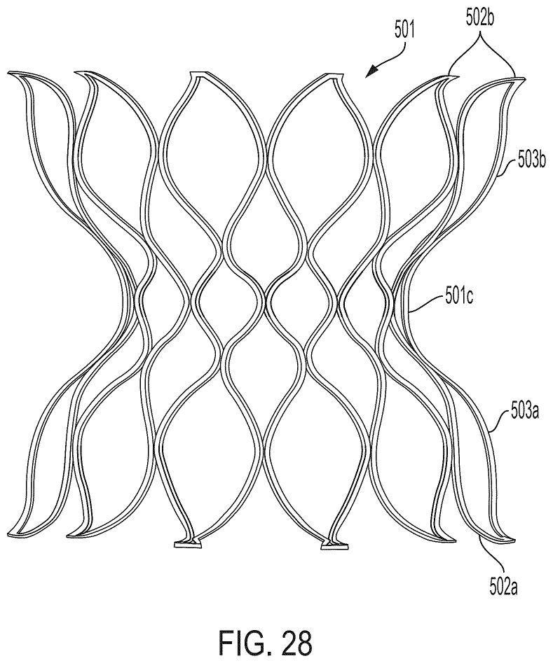

In some examples, as shown in FIG. 27, an expandable stent or docking station 400 includes an exterior cover or skirt 406 covering at least a tissue engaging portion (e.g., convex sealing portions 403a, 403b) of a docking station frame 401, to provide a layer of cushioning between the docking station frame and the tissue at the implant site. As shown, the docking station frame 401 may be similar to the docking station frame 1500-1 of FIGS. 17A and 17B. In some examples, a similar cover or skirt configuration may be applied to any suitable docking station frame, including, for example, the docking station frames shown in FIGS. 18A-23B and described above.

The exterior cover 406 may be provided in a substantially impermeable material selected to provide sufficient cushioning (e.g., hardness rating of about 75 Shore A) and impermeability, such as, for example, TPU, including carbonate based, biocompatible, unmodified, modified siloxinated, and/or fluorinated TPU.

The docking station frame 401 can have an inflow end portion 401a, an outflow end portion 401b, and a central waist portion 401c. In the illustrated example, the exterior cover 406 is positioned so as not to cover at least portions of the outflow end portion 401b of the docking station frame 401 (e.g., to limit cushioning to portions having substantial sealing contact with the implant site), with the cover material being truncated axially inward of the outflow end portion, around an axial midpoint of the distal and proximal rows of cells of the docking station. In some examples, the exterior cover material 406 may be truncated at a different location along the length of the cover material (e.g., along the axially inner horizontal strut(s) of the proximal and/or distal rows of cells 405a, 405b of the docking station frame 401), or may extend over the entire axial length of the docking station frame (e.g., with openings or cutouts aligned with the distal row of cells, as described above and schematically shown in FIG. 9C).

In the illustrated example, the docking station 400 includes an inner material 407 extending from a proximal end of the docking station frame 401 to the axially inner horizontal strut(s) of the distal row of cells 405b, for example, for seating and/or scaling contact with an implanted medical device (e.g., prosthetic valve) and to provide additional scaling against the pressurized blood at the inflow end 401a of the docking station. The inner material 407 may be provided in any suitable material (e.g., PET) and may have a thickness (e.g., between about 0.1 mm and about 0.15 mm) sufficient for sealing retention of a catheter installed and expanded prosthetic valve or other such implanted medical device, and may be substantially impermeable, for example, to inhibit tissue ingrowth into the inner diameter of the docking station frame 401.

The frame of a docking station or other such stent (e.g., any of the stent frames of FIGS. 17A-23B) can be formed from wires or a lattice. The frame can be self-expanding, manually expandable (e.g., expandable via balloon), or mechanically expandable. A self-expanding frame can be made of a shape memory material such as, for example, nitinol.

According to another exemplary aspect of the present disclosure, an expandable docking station may be provided with a lattice frame having a rounded scaling profile in the fully expanded condition, for example, to provide an elongated, softened seal zone when the frame apices are bent radially inward by engagement with the vessel tissue at the implant site in a deployed condition of the docking station. This rounded sealing profile may more evenly distribute sealing forces between the docking station and the implant site tissue, for example, to mitigate damage and erosion of the tissue.

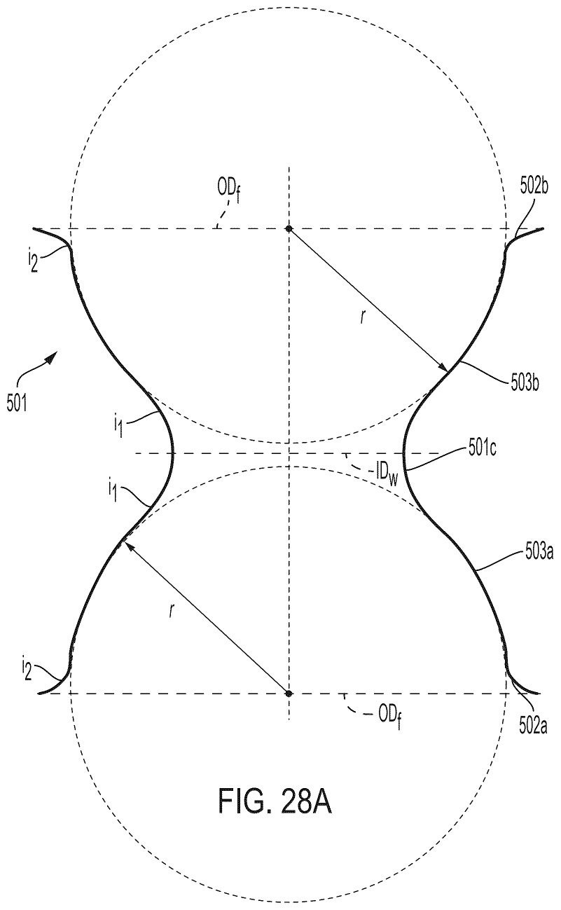

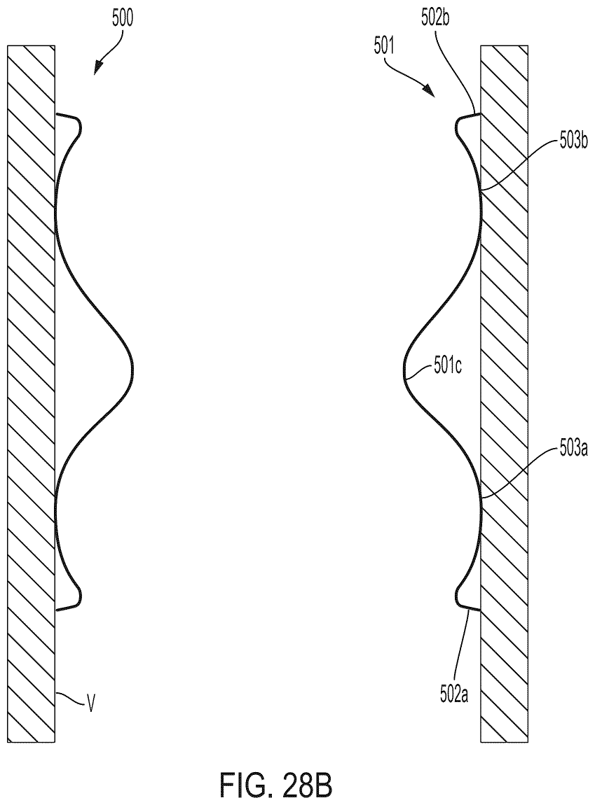

In some examples, as shown in FIGS. 28, 28A and 28B, an expandable stent or docking station frame 501 may include an hourglass shaped profile (for example, as shown in FIGS. 17A and 17B) with flared end (retaining) portions 502a, 502b, convex medial (scaling) portions 503a, 503b, and a concave central waist portion 501c. As shown in FIG. 28A, the convex medial sealing portions 503a, 503b may have a substantially uniform axially extending rounded contour in profile (i.e., substantially constant radius of curvature r) extending from the concave waist portion 501c (at inflection points i₁) to the flared retaining portions (at inflection points 12). In some examples, when the docking station frame 501 is deployed in a vessel V (FIG. 28B), the radial inward bending/constriction of the frame at the flared ends 502a, 502b produces an elongated or softened sealing or contact region between the docking station 500 and the implant site tissue. Due to the substantially uniform contour curvature, this elongated or softened sealing or contact region may be maintained over a range of vessel sizes (and resulting frame constrictions). In some examples, the inflection points i₂ between the sealing portions 503a, 503b and the flared retaining portions 502a, 502b may be positioned at a maximum diameter of the sealing portions when the frame 501 is in the fully expanded condition, such that there are no radially inward extending portions between the sealing portions and the retaining portions. In some examples, an expanded docking station frame 501 includes a waist portion 501c having an inner diameter ID_{w} of between about 25 mm and about 29 mm, flared retaining portions 502a, 502b having an outer diameter OD_{f} between about 44 mm and about 48 mm, and scaling portions 503a, 503b having a uniform radius of curvature r of approximately 11.5 mm.

According to another exemplary aspect of the present disclosure, an expandable docking station may additionally or alternatively be provided with a polymer coating applied to the frame struts, for example, to minimize or mitigate damage and erosion of the tissue at the implant site. The coating may be selected to provide reduced hardness (e.g., about 75 to about 90 Shore A hardness) of the frame and/or reduced friction between the docking station frame and the implant site tissue. The coating may be applied over the entire docking station frame, or the coating may be limited to one or more of the tissue engaging portions of the frame (e.g., flared retaining portions and/or convex scaling portions).

The polymer coating may be provided in a variety of suitable materials, including, for example, parylene and urethane (e.g., thermoplastic polyurethanes, such as biocompatible, unmodified, modified, siloxinated, and fluorinated polyurethanes). The polymer coating may be provided in a variety of suitable thicknesses, for example, between about 0.5 micron and about 4.0 micron thickness.

The expandable stents described herein may form a docking station for securely retaining an implantable medical device (e.g., an expandable prosthetic valve) within a vessel of a subject. In some examples, the expandable stent may be provided with an integrally retained medical device, such as, for example, a plurality of leaflets directly fixed to the inner periphery of the expandable stent to form an expandable prosthetic valve.

A prosthetic valve used with an expandable stent or docking station 10, functioning as a docking station, can take a wide variety of different forms. In some examples, the valve is configured to be implanted via a catheter in the heart. For example, the valve can be expandable and collapsible to facilitate transcatheter application in a heart. However, in some examples, the valve can be configured for surgical application. Similarly, the docking stations described herein can be placed using transcatheter application/placement or surgical application/placement.

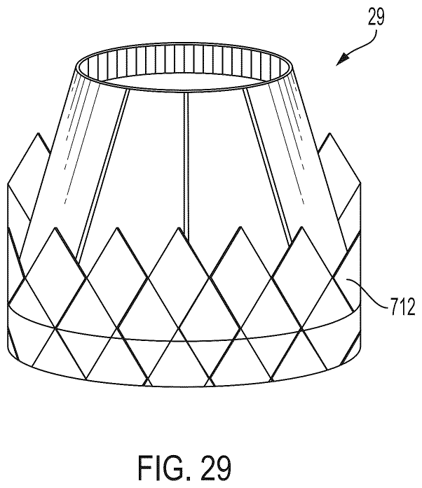

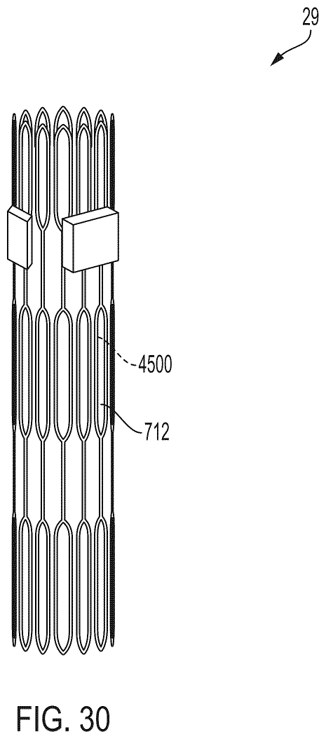

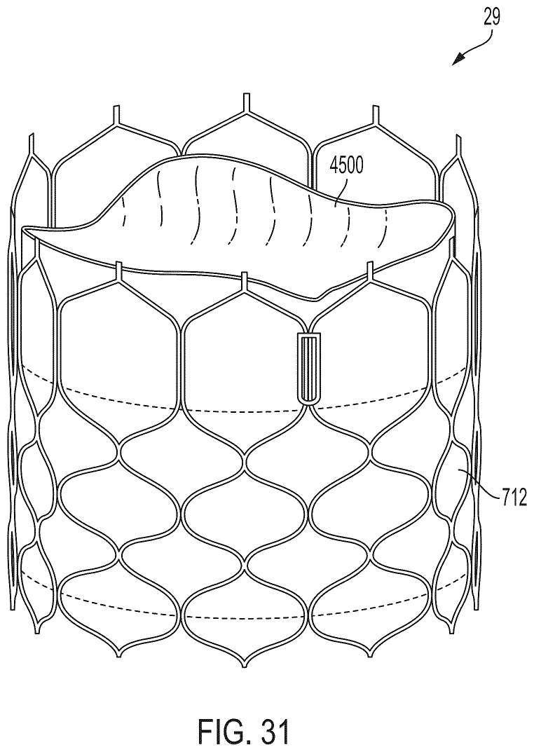

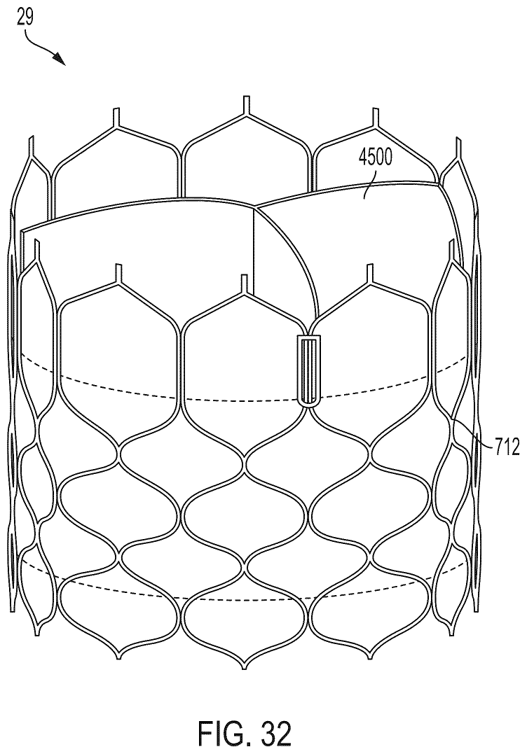

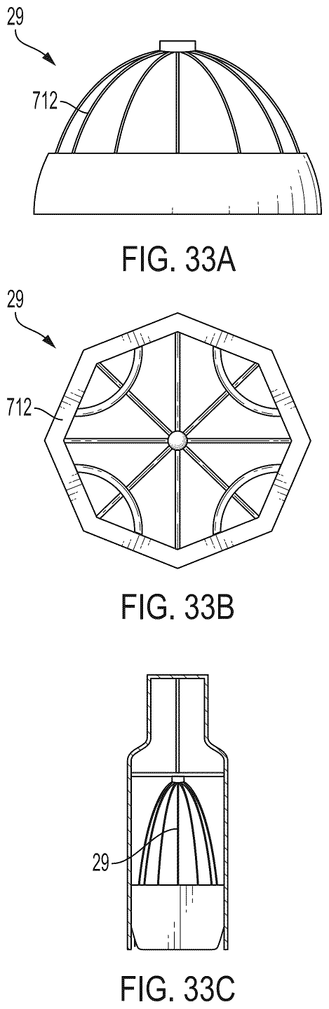

FIGS. 29-33C illustrate a few examples of the many valves or valve configurations that can be used. Any valve type can be used and some valves that are traditionally applied surgically can be modified for transcatheter implantation. FIG. 29 illustrates an expandable valve 29 for transcatheter implantation that is shown and described in U.S. Pat. No. 8,002,825, which is incorporated herein by reference in its entirety. An example of a tri-leaflet valve is shown and described in Published Patent Cooperation Treaty Application No. WO 2000/42950, which is incorporated herein by reference in its entirety. Another example of a tri-leaflet valve is shown and described in U.S. Pat. No. 5,928,281, which is incorporated herein by reference in its entirety. Another example of a tri-leaflet valve is shown and described in U.S. Pat. No. 6,558,418, which is incorporated herein by reference in its entirety. FIGS. 29-32 illustrate some examples of an expandable tri-leaflet valve 29, such as the Edwards SAPIEN Transcatheter Heart Valve. In some examples, as shown in FIG. 30, the valve 29 comprises a frame 712 that contains a tri-leaflet valve 4500 (see FIG. 31) compressed inside the frame 712. FIG. 31 illustrates the frame 712 expanded and the valve 29 in an open condition. FIG. 32 illustrates the frame 712 expanded and the valve 29 in a closed condition. FIGS. 33A, 33B, and 33C illustrate an example of an expandable valve 29 that is shown and described in U.S. Pat. No. 6,540,782, which is incorporated herein by reference in its entirety. An example of a valve is shown and described in U.S. Pat. No. 3,365,728, which is incorporated herein by reference in its entirety. Another example of a valve is shown and described in U.S. Pat. No. 3,824,629, which is incorporated herein by reference in its entirety. Another example of a valve is shown and described in U.S. Pat. No. 5,814,099, which is incorporated herein by reference in its entirety. Any of these or other valves can be used as valve 29 in the various examples disclosed herein.

Methods of treating a patient (e.g., methods of treating heart valve dysfunction/regurgitation, etc.), or carrying out a simulation of treating a patient, can include a variety of steps, including steps associated with introducing and deploying a docking station in a desired location/treatment area and introducing and deploying a valve in the docking station.

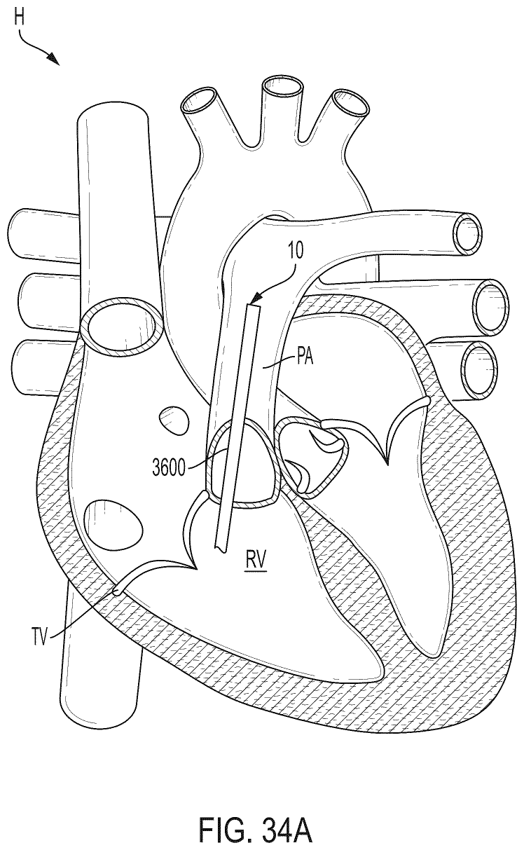

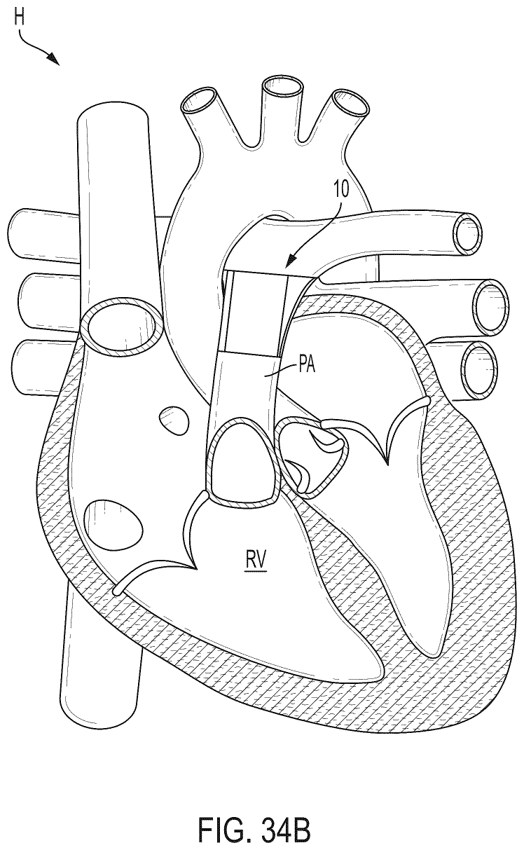

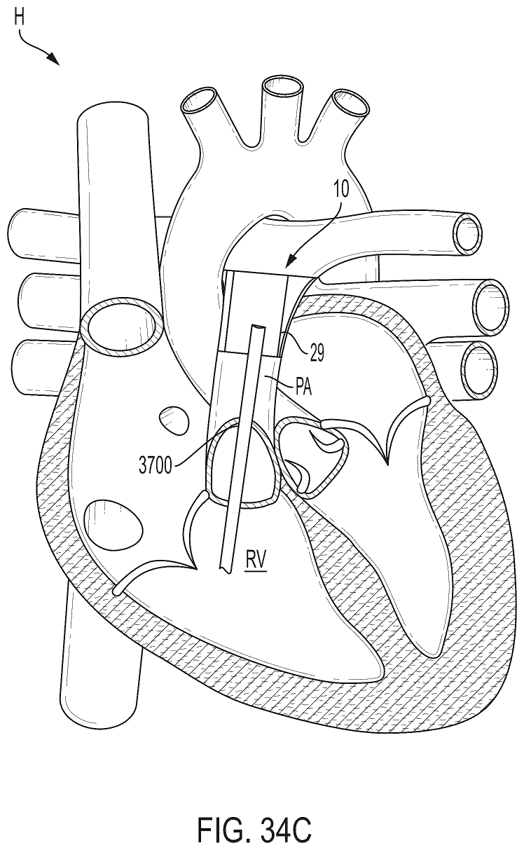

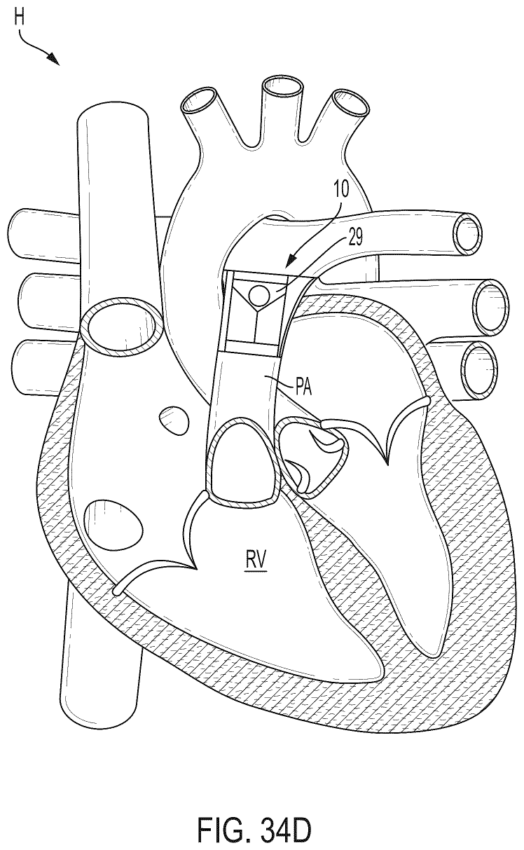

For example, FIG. 34A illustrates a docking station 10 (e.g., any of the docking stations utilizing any of the docking station frames 1500 described herein) being deployed by a first catheter 3600 including a first tube retaining the docking station prior to deployment. In some examples, a guidewire followed by a catheter 3600 is advanced to the pulmonary artery PA by way of the femoral vein, inferior vena cava, tricuspid valve, and right ventricle RV. When deployed, as shown in FIG. 34B, the docking station frame 1500 expands at the target location, with the proximal first end of the docking station frame expanding radially outward of the valve seat to engage the inner surface of the pulmonary artery PA, to retain the docking station frame at the target location. The first catheter is then withdrawn from the pulmonary artery. A second catheter 3700 with a second tube retaining an expandable prosthetic valve in a compressed condition is then inserted into the pulmonary artery PA. A terminal end of the second tube is extended into the first end of the expanded docking station frame (FIG. 34C). The expandable prosthetic valve 29 is deployed from the terminal end of the second tube and expanded into seating engagement with the valve seat 18 of the expanded docking station frame (FIG. 34D).

The foregoing primarily describes examples of expandable stents that are self-expanding. But the docking stations and/or delivery devices shown and described herein can be modified for delivery of balloon-expandable and/or mechanically-expandable devices, within the scope of the present disclosure. That is to say, delivering balloon-expandable and/or mechanically-expandable docking stations to an implantation location can be performed percutaneously using modified versions of the delivery devices of the present disclosure. In general terms, this includes providing a transcatheter assembly that can include a delivery sheath and/or additional sheaths as described above. In the case of balloon-expandable docking stations, the devices generally further include a delivery catheter, a balloon catheter, and/or a guide wire. A delivery catheter used in a balloon-expandable type of delivery device can define a lumen within which the balloon catheter is received. The balloon catheter, in turn, defines a lumen within which the guide wire is slidably disposed. Further, the balloon catheter includes a balloon that is fluidly connected to an inflation source. With the docking station mounted on the balloon, the transcatheter assembly is delivered through a percutaneous opening in the patient via the delivery device. Once the docking station is properly positioned, the balloon catheter is operated to inflate the balloon, thus transitioning the docking station to an expanded arrangement.

### EXAMPLES

Example 1. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent.

Example 2. The expandable stent of Example 1, wherein the outer material comprises a permeable material and the inner material comprises an impermeable material.

Example 3. The expandable stent of any of Examples 1-2, wherein at least a portion of the outer material defines a tissue engaging retaining portion.

Example 4. The expandable stent of any of Examples 1-3, wherein at least a portion of the inner material defines a valve seat.

Example 5. The expandable stent of any of Examples 1-4, wherein at least a portion of the outer material is axially aligned with at least a portion of the inner material.

Example 6. The expandable stent of Example 5, wherein the axially aligned portions of the outer material and inner material encapsulate at least a portion of the expandable frame.

Example 7. The expandable stent of any of Examples 1-6, wherein at least a portion of the outer material is directly attached to the expandable frame.

Example 8. The expandable stent of Example 7, wherein the at least a portion of the outer material is attached to the expandable frame by an adhesive.

Example 9. The expandable stent of Example 7, wherein the at least a portion of the outer material is sutured to the expandable frame.

Example 10. The expandable stent of Example 7, wherein the at least a portion of the outer material is attached to the expandable frame through heat fixation.

Example 11. The expandable stent of any of Examples 1-10, wherein at least a portion of the inner material is directly attached to the expandable frame.

Example 12. The expandable stent of Example 11, wherein the at least a portion of the inner material is attached to the expandable frame by an adhesive.

Example 13. The expandable stent of Example 11, wherein the at least a portion of the inner material is sutured to the expandable frame.

Example 14. The expandable stent of Example 11, wherein the at least a portion of the inner material is attached to the expandable frame through heat fixation.

Example 15. The expandable stent of any of Examples 1-14, wherein at least a portion of the outer material is directly attached to at least a portion of the inner material.

Example 16. The expandable stent of Example 15, wherein the at least a portion of the outer material is attached to the at least a portion of the inner material by an adhesive.

Example 17. The expandable stent of Example 15, wherein the at least a portion of the outer material is sutured to the at least a portion of the inner material.

Example 18. The expandable stent of Example 15, wherein the at least a portion of the outer material is attached to the at least a portion of the inner material through heat fixation.

Example 19. The expandable stent of any of Examples 1-18, wherein the outer material comprises a shrink-wrap material.

Example 20. The expandable stent of any of Examples 1-19, wherein the inner material comprises a shrink-wrap material.

Example 21. The expandable stent of any of Examples 1-20, wherein the outer material comprises at least one of a knitted material, a woven material, and a perforated material.

Example 22. The expandable stent of any of Examples 1-21, wherein the outer material comprises at least one of polyethylene terephthalate (PET), fluorinated ethylene propylene (FEP), polytetrafluoroethylene (PTFE), ethylene tetrafluoroethylene (ETFE), and thermoplastic polyurethane (TPU).

Example 23. The expandable stent of any of Examples 1-22, wherein the inner material comprises at least one of polyethylene terephthalate (PET), fluorinated ethylene propylene (FEP), polytetrafluoroethylene (PTFE), ethylene tetrafluoroethylene (ETFE), and thermoplastic polyurethane (TPU).

Example 24. The expandable stent of any of Examples 1-23, wherein the expandable frame comprises a plurality of struts.

Example 25. The expandable stent of Example 24, wherein the plurality of struts defines axially endmost apices, wherein the outer material covers exterior surfaces of the axially endmost apices.

Example 26. The expandable stent of any of Examples 24-25, wherein the plurality of struts defines a plurality of cells, wherein a portion of the plurality of cells is at least partially uncovered by the inner material.

Example 27. The expandable stent of Example 26, wherein the portion of the plurality of cells is at least partially uncovered by both the outer material and the inner material.

Example 28. The expandable stent of Example 27, wherein the at least partially uncovered portion of the plurality of cells is defined by strut portions that are covered by the outer material.

Example 29. The expandable stent of any of Examples 26-28, wherein the at least partially uncovered portion of the plurality of cells comprises distalmost cells of the plurality of cells.

Example 30. The expandable stent of any of Examples 1-29, further comprising at least one radiopaque marker encapsulated between the first and inner materials.

Example 31. The expandable stent of Example 30, wherein at least a portion of the inner material defines a valve seat, and wherein the at least one radiopaque marker is axially aligned with the valve seat.

Example 32. The expandable stent of any of Examples 1-31, wherein the inner material comprises first and second layers, wherein at least a portion of the first layer defines a valve seat.

Example 33. The expandable stent of any of Examples 1-32, wherein the expandable frame comprises a self-expanding frame.

Example 34. The expandable stent of any of Examples 1-33, wherein the expandable stent is a docking station, and wherein the inner periphery is configured to retain an expandable medical device.

Example 35. The expandable stent of any of Examples 1-33, wherein the expandable stent is a docking station, and wherein the inner periphery is configured to retain an expandable prosthetic valve.

Example 36. The expandable stent of any of Examples 1-35, wherein the inner material has a thickness between about 0.1 mm and about 0.15 mm.

Example 37. The expandable stent of any of Examples 1-36, wherein the outer material comprises a biodegradable or bioresorbable polymeric material.

Example 38. The expandable stent of Example 37, wherein the biodegradable or bioresorbable polymeric material comprises at least one of poly-l-lactic acid (PLLA), polycaprolactone (PCL), and poly(4-hydroxybutyrate) (P4HB).

Example 39. The expandable stent of any of Examples 1-38, wherein the outer material has a thickness between about 0.05 mm and about 0.07 mm.

Example 40. The expandable stent of any of Examples 1-39 wherein the outer material comprises a woven material formed from yarns having a linear density between about 20 decitex and about 25 decitex.

Example 41. The expandable stent of any of Examples 1-40 wherein the outer material comprises a woven material formed from yarns having a thread density between about 160 picks per inch and about 225 picks per inch.

Example 42. The expandable stent of any of Examples 1-38, wherein the outer material has a thickness between about 0.2 mm and about 0.6 mm.

Example 43. The expandable stent of Example 42, wherein the outer material is configured to experience a reduction in thickness of up to between about 15% and about 20% under compression in a deployed condition of the expandable stent.

Example 44. The expandable stent of any of Examples 42-43, wherein the outer material has a stretchability between about 40% and about 60%.

Example 45. The expandable stent of any of Examples 42-44, wherein the outer material comprises at least one of a knitted polymer material, a polymer foam, and a nonwoven textile material.

Example 46. The expandable stent of any of Examples 1-36, wherein the outer material comprises at least one of carbonate based, biocompatible, unmodified siloxinated, modified siloxinated, and fluorinated thermoplastic polyurethane (TPU).

Example 47. The expandable stent of Example 46, wherein the outer material has a thickness between about 0.07 mm and about 0.20 mm.

Example 48. The expandable stent of any of Examples 1-38, wherein the outer material comprises a bidirectionally stretchable cloth material.

Example 49. The expandable stent of Example 48, wherein the bidirectionally stretchable cloth material comprises honeycomb cloth.

Example 50. The expandable stent of Example 48, wherein the outer material comprises a textured crochet knit material.

Example 51. The expandable stent of any of Examples 48-50, wherein the outer material has a thickness between about 0.40 mm and about 0.60 mm.

Example 52. The expandable stent of any of Examples 48-51, wherein the outer material has a stretchability between about 60% and about 100% in both an axial direction and a circumferential direction.

Example 53. An expandable frame comprising a plurality of struts defining a plurality of cells, the expandable frame having an hourglass shaped profile when in an expanded condition, with flared endmost retaining portions, convex medial sealing portions, and a concave central waist portion, wherein the convex medial sealing portions have a substantially uniform axially extending rounded contour in profile extending from the concave waist portion to the flared retaining portions.

Example 54. The expandable frame of Example 53, wherein the sealing portions and the flared retaining portions are joined at inflection points positioned at a maximum diameter of the sealing portions when the frame is in the fully expanded condition, such that there are no radially inward extending portions between the sealing portions and the retaining portions.

Example 55. The expandable frame of any of Examples 53-54, wherein the scaling portions have a uniform radius of curvature of approximately 11.5 mm.

Example 56. The expandable frame of any of Examples 53-55, further comprising a polymer coating applied to the plurality of struts at least at the sealing portions.

Example 57. The expandable frame of Example 56, wherein the polymer coating comprises at least one of parylene and thermoplastic polyurethane (TPU).

Example 58. The expandable frame of any of Example 56-57, wherein the polymer coating has a hardness between about 75 Shore A and about 90 Shore A.

Example 59. The expandable frame of any of Examples 56-58, wherein the polymer coating has a thickness between about 0.5 micron and about 4.0 micron.

Example 60. An expandable frame comprising a plurality of struts defining a plurality of cells, and a polymer coating applied to at least a radially outermost sealing portion of the plurality of struts.

Example 61. The expandable frame of Example 60, wherein the polymer coating comprises at least one of parylene and thermoplastic polyurethane (TPU).

Example 62. The expandable frame of any of Examples 60-61, wherein the polymer coating has a hardness between about 75 Shore A and about 90 Shore A.

Example 63. The expandable frame of any of Examples 60-62, wherein the polymer coating has a thickness between about 0.5 micron and about 4.0 micron.

Example 64. The expandable stent of any of Example 1-52, wherein the expandable frame comprises the expandable frame of any of Example 53-63.

Example 65. A prosthetic valve assembly comprising the expandable stent of any of Examples 1-52 and 64; and a prosthetic valve secured to a valve seat of the expandable stent.

Example 66. The prosthetic valve assembly of Example 65, wherein the inner material sealingly engages the prosthetic valve.

Example 67. The prosthetic valve assembly of any of Examples 65-66, wherein the prosthetic valve comprises an expandable frame.

Example 68. A system comprising: a catheter including a sleeve; and the expandable stent of any of Examples 1-52 and 64, wherein the expandable stent is disposed in the sleeve in an unexpanded condition.

Example 69. A method of manufacturing an expandable stent, the method comprising: providing an expandable frame extending axially from a proximal end to a distal end; attaching an outer material to an exterior of the expandable frame to define an outer periphery of the expandable stent; and attaching an inner material to an interior of the expandable frame to define an inner periphery of the expandable stent.

Example 70. The method of Example 69, wherein the outer material comprises a permeable material and the inner material comprises an impermeable material.

Example 71. The method of any of Examples 69-70, wherein at least a portion of the outer material defines a tissue engaging retaining portion.

Example 72. The method of any of Examples 69-71, wherein at least a portion of the inner material defines a valve seat.

Example 73. The method of any of Examples 69-72, wherein attaching the first and inner materials to the expandable frame comprises axially aligned at least a portion of the outer material with at least a portion of the inner material.

Example 74. The method of any of Examples 69-73, wherein attaching the first and inner materials to the expandable frame comprises encapsulating at least a portion of the expandable frame between axially aligned portions of the first and inner materials.

Example 75. The method of any of Examples 69-74, wherein attaching the outer material to the exterior of the expandable frame comprises directly attaching at least a portion of the outer material to the expandable frame.

Example 76. The method of Example 75, wherein directly attaching the at least a portion of the outer material to the expandable frame comprises attaching the at least a portion of the outer material to the expandable frame by an adhesive.

Example 77. The method of Example 75, wherein directly attaching the at least a portion of the outer material to the expandable frame comprises suturing the at least a portion of the outer material to the expandable frame.

Example 78. The method of Example 75, wherein directly attaching the at least a portion of the outer material to the expandable frame comprises heat fixing the at least a portion of the outer material to the expandable frame.

Example 79. The method of any of Examples 69-78, wherein attaching the inner material to the interior of the expandable frame comprises directly attaching at least a portion of the inner material to the expandable frame.

Example 80. The method of Example 79, wherein directly attaching the at least a portion of the inner material to the expandable frame comprises attaching the at least a portion of the inner material to the expandable frame by an adhesive.

Example 81. The method of Example 79, wherein directly attaching the at least a portion of the inner material to the expandable frame comprises suturing the at least a portion of the inner material to the expandable frame.

Example 82. The method of Example 79, wherein directly attaching the at least a portion of the inner material to the expandable frame comprises heat fixing the at least a portion of the inner material to the expandable frame.

Example 83. The method of any of Examples 69-82, wherein attaching the first and inner materials to the expandable frame comprises directly attaching at least a portion of the outer material to at least a portion of the inner material.

Example 84. The method of Example 83, wherein directly attaching the at least a portion of the outer material to the at least a portion of the inner material comprises attaching the at least a portion of the outer material to the at least a portion of the inner material by an adhesive.

Example 85. The method of Example 83, wherein directly attaching the at least a portion of the outer material to the at least a portion of the inner material comprises suturing the at least a portion of the outer material to the at least a portion of the inner material.

Example 86. The method of Example 83, wherein directly attaching the at least a portion of the outer material to the at least a portion of the inner material comprises heat fixing the at least a portion of the outer material to the at least a portion of the inner material.

Example 87. The method of any of Examples 69-86, wherein the outer material comprises a tube of shrink-wrap material, wherein attaching the outer material to the exterior of the expandable frame comprises sliding the tube of shrink-wrap material over the expandable frame and shrinking the tube of shrink-wrap material against the exterior of the expandable frame.

Example 88. The method of any of Examples 69-87, wherein the inner material comprises a tube of shrink-wrap material, wherein attaching the inner material to the interior of the expandable frame comprises sliding the tube of shrink-wrap material into the expandable frame and expanding the tube of shrink-wrap material against the interior of the expandable frame.

Example 89. The method of any of Examples 69-88, further comprising thermoforming the outer material to a contoured shape corresponding to an exterior contoured surface of the expandable frame.

Example 90. The method of any of Examples 69-89, further comprising thermoforming the inner material to a contoured shape corresponding to an interior contoured surface of the expandable frame.

Example 91. The method of any of Examples 69-86, wherein attaching the outer material to the exterior of the expandable frame comprises wrapping a layer of the outer material around the exterior of the expandable frame.

Example 92. The method of any of Examples 69-86 and 91, wherein attaching the inner material to the interior of the expandable frame comprises wrapping a layer of the outer material around the interior of the expandable frame.

Example 93. The method of any of Examples 69-92, wherein attaching the outer material to the exterior of the expandable frame comprises covering exterior surfaces of axially endmost apices of the expandable frame with the outer material.

Example 94. The method of any of Examples 69-93, wherein the expandable frame comprises a plurality of struts defining a plurality of cells, wherein attaching the inner material to the interior of the expandable frame comprises leaving a portion of the plurality of cells at least partially uncovered by the inner material.

Example 95. The method of Example 94, wherein attaching the outer material to the exterior of the expandable frame comprises leaving the portion of the plurality of cells at least partially uncovered by both the outer material and the inner material.

Example 96. The method of Example 95, wherein the at least partially uncovered portion of the plurality of cells is defined by strut portions that are covered by the outer material.

Example 97. The method of any of Examples 94-96, wherein the at least partially uncovered portion of the plurality of cells comprises distalmost cells of the plurality of cells.

Example 98. The method of any of Examples 69-97, further comprising encapsulating at least one radiopaque marker between the first and inner materials.

Example 99. The method of Example 98, wherein at least a portion of the inner material defines a valve seat, and wherein the at least one radiopaque marker is axially aligned with the valve seat.

Example 100. The method of any of Examples 69-99, wherein the inner material comprises first and second layers, wherein at least a portion of the first layer defines a valve seat.

Example 101. The method of any of claims 69-100, wherein the expandable frame comprises a self-expanding frame.

Example 102. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the inner material has a thickness between about 0.1 mm and about 0.15 mm.

Example 103. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises a biodegradable or bioresorbable polymeric material.

Example 104. The expandable stent of Example 103, wherein the biodegradable or bioresorbable polymeric material comprises at least one of poly-1-lactic acid (PLLA), polycaprolactone (PCL), and poly(4-hydroxybutyrate) (P4HB).

Example 105. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material has a thickness between about 0.05 mm and about 0.07 mm.

Example 106. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises a woven material formed from yarns having a linear density between about 20 decitex and about 25 decitex.

Example 107. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises a woven material formed from yarns having a thread density between about 160 picks per inch and about 225 picks per inch.

Example 108. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material has a thickness between about 0.2 mm and about 0.6 mm.

Example 109. The expandable stent of Example 108, wherein the outer material is configured to experience a reduction in thickness of up to between about 15% and about 20% under compression in a deployed condition of the expandable stent.

Example 110. The expandable stent of any of Examples 108-109, wherein the outer material has a stretchability between about 40% and about 60%.

Example 111. The expandable stent of any of claims 7-9, wherein the outer material comprises at least one of a knitted polymer material, a polymer foam, and a nonwoven textile material.

Example 112. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises at least one of carbonate based, biocompatible, unmodified siloxinated, modified siloxinated, and fluorinated thermoplastic polyurethane (TPU).

Example 113. The expandable stent of Example 112, wherein the outer material has a thickness between about 0.07 mm and about 0.20 mm.

Example 114. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises a bidirectionally stretchable cloth material.

Example 115. The expandable stent of Example 114, wherein the bidirectionally stretchable cloth material comprises honeycomb cloth.

Example 116. The expandable stent of Example 114, wherein the outer material comprises a textured crochet knit material.

Example 117. The expandable stent of any of Examples 114-116, wherein the outer material has a thickness between about 0.40 mm and about 0.60 mm.

Example 118. The expandable stent of any of Examples 114-117, wherein the outer material has a stretchability between about 60% and about 100% in both an axial direction and a circumferential direction.

Example 119. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the outer material comprises polyethylene terephthalate (PET).

Example 120. An expandable stent comprising: an expandable frame extending axially from a proximal end to a distal end; an outer material attached to an exterior of the expandable frame to define an outer periphery of the expandable stent; and an inner material attached to an interior of the expandable frame to define an inner periphery of the expandable stent; wherein the inner material comprises polyethylene terephthalate (PET).

Example 121. The expandable stent of any of Examples 102-120, wherein the outer material comprises a permeable material and the inner material comprises an impermeable material.

Example 122. The expandable stent of any of Examples 102-121, wherein at least a portion of the outer material defines a tissue engaging retaining portion.

Example 123. The expandable stent of any of Examples 102-122, wherein at least a portion of the inner material defines a valve seat.

Example 124. The expandable stent of any of Examples 102-123, wherein at least a portion of the outer material is axially aligned with at least a portion of the inner material.

Example 125. The expandable stent of Example 124, wherein the axially aligned portions of the outer material and inner material encapsulate at least a portion of the expandable frame.

Example 126. The expandable stent of any of Examples 102-125, wherein at least a portion of the outer material is directly attached to the expandable frame.

Example 127. The expandable stent of Example 126, wherein the at least a portion of the outer material is attached to the expandable frame by an adhesive.

Example 128. The expandable stent of Example 126, wherein the at least a portion of the outer material is sutured to the expandable frame.

Example 129. The expandable stent of Example 126, wherein the at least a portion of the outer material is attached to the expandable frame through heat fixation.

Example 130. The expandable stent of any of Examples 102-129, wherein at least a portion of the inner material is directly attached to the expandable frame.

Example 131. The expandable stent of Example 130, wherein the at least a portion of the inner material is attached to the expandable frame by an adhesive.

Example 132. The expandable stent of Example 130, wherein the at least a portion of the inner material is sutured to the expandable frame.

Example 133. The expandable stent of Example 130, wherein the at least a portion of the inner material is attached to the expandable frame through heat fixation.

Example 134. The expandable stent of any of Examples 102-133, wherein at least a portion of the outer material is directly attached to at least a portion of the inner material.

Example 135. The expandable stent of Example 134, wherein the at least a portion of the outer material is attached to the at least a portion of the inner material by an adhesive.

Example 136. The expandable stent of Example 134, wherein the at least a portion of the outer material is sutured to the at least a portion of the inner material.

Example 137. The expandable stent of Example 134, wherein the at least a portion of the outer material is attached to the at least a portion of the inner material through heat fixation.

Example 138. The expandable stent of any of Examples 102-137, wherein the expandable frame comprises a plurality of struts.

Example 139. The expandable stent of Example 138, wherein the plurality of struts defines a plurality of cells, wherein a portion of the plurality of cells is at least partially uncovered by the inner material.

Example 140. The expandable stent of Example 139, wherein the portion of the plurality of cells is at least partially uncovered by both the outer material and the inner material.

Example 141. The expandable stent of any of Examples 139-140, wherein the at least partially uncovered portion of the plurality of cells comprises distalmost cells of the plurality of cells.

Example 142. The expandable stent of any of Examples 102-141, wherein at least a portion of the inner material defines a valve seat, and wherein at least one radiopaque marker is axially aligned with the valve seat.

Example 143. The expandable stent of any of Examples 102-142, wherein the expandable frame comprises a self-expanding frame.

Example 144. The expandable stent of any of Examples 102-143, wherein the expandable stent is a docking station, and wherein the inner periphery is configured to retain an expandable medical device.

Example 145. The expandable stent of any of Examples 102-144, wherein the expandable stent is a docking station, and wherein the inner periphery is configured to retain an expandable prosthetic valve.

In view of the many possible examples to which the principles of the disclosed disclosure may be applied, it should be recognized that the illustrated arrangements are only examples of the disclosure and should not be taken as limiting the scope of the disclosure. All combinations or sub-combinations of features of the foregoing examples are contemplated by this disclosure.

While various inventive aspects, concepts and features of the disclosure may be described and illustrated herein as embodied in combination in disclosed examples, these various aspects, concepts and features may be used in many alternative examples, either individually or in various combinations and sub-combinations thereof. Unless expressly excluded herein all such combinations and sub-combinations are intended to be within the scope of the present inventions. Still further, while various alternative embodiments as to the various aspects, concepts and features of the disclosure—such as alternative materials, structures, configurations, methods, devices and components, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the disclosure may be described herein as being an exemplary arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to at least include the specified value, values within 5% of the specified value, and values within 10% of the specified value, unless expressly stated otherwise. Further, it is to be understood that the drawings accompanying the present disclosure may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

What is claimed is:

1. An expandable frame comprising a plurality of struts defining a plurality of cells, the expandable frame having an hourglass shaped profile when in an expanded condition, with flared endmost retaining portions, convex medial sealing portions, and a concave central waist portion, wherein the convex medial sealing portions have a substantially uniform axially extending rounded contour in profile extending from the concave central waist portion to the flared endmost retaining portions.

2. The expandable frame of claim 1, wherein the sealing portions and the flared endmost retaining portions are joined at inflection points positioned at a maximum diameter of the sealing portions when the expandable frame is in a fully expanded condition, such that there are no radially inward extending portions between the sealing portions and the retaining portions.

3. An expandable frame comprising a plurality of struts defining a plurality of cells, and a polymer coating applied to at least a radially outermost sealing portion of the plurality of struts.

4. The expandable frame of claim 3, wherein the polymer coating comprises at least one of parylene and thermoplastic polyurethane (TPU).

5. The expandable frame of claim 3, wherein the polymer coating has at least one of: a hardness between about 75 Shore A and about 90 Shore A, and a thickness between about 0.5 micron and about 4.0 micron.

6. An expandable stent comprising:
an expandable frame extending axially from a proximal end to a distal end;
an outer material attached to an exterior of the expandable frame to define an outer periphery of the expandable stent; and
an inner material attached to an interior of the expandable frame to define an inner periphery of the expandable stent.

7. The expandable stent of claim 6, wherein the outer material comprises a permeable material and the inner material comprises an impermeable material.

8. The expandable stent of claim 6, wherein at least a portion of the outer material is axially aligned with at least a portion of the inner material, and wherein the axially aligned portions of the outer material and the inner material encapsulate at least a portion of the expandable frame.

9. The expandable stent of claim 6, wherein the outer material comprises at least one of: a shrink-wrap material, a thermoformed material, a knitted material, a woven material, a perforated material, polyethylene terephthalate (PET), fluorinated ethylene propylene (FEP), polytetrafluoroethylene (PTFE), ethylene tetrafluoroethylene (ETFE), thermoplastic polyurethane (TPU), a biodegradable material, a bioresorbable polymeric material, poly-1-lactic acid (PLLA), polycaprolactone (PCL), poly(4-hydroxybutyrate) (P4HB), a knitted polymer material, a polymer foam, a nonwoven textile material, a bidirectionally stretchable cloth material, a honeycomb cloth, a textured crochet knit material, a carbonate based material, a biocompatible material, an unmodified siloxinated material, a modified siloxinated material, and fluorinated TPU.

10. The expandable stent of claim 6, wherein the inner material comprises at least one of a shrink-wrap material, a thermoformed material, polyethylene terephthalate (PET), fluorinated ethylene propylene (FEP), polytetrafluoroethylene (PTFE), ethylene tetrafluoroethylene (ETFE), thermoplastic polyurethane (TPU), and a dual layer material.

11. The expandable stent of claim 6, wherein the expandable frame comprises a plurality of struts defining axially endmost apices, wherein the outer material covers exterior surfaces of the axially endmost apices.

12. The expandable stent of claim 6, wherein the expandable frame comprises a plurality of struts defining a plurality of cells, wherein a portion of the plurality of cells is at least partially uncovered by the inner material.

13. The expandable stent of any of claim 6, further comprising at least one radiopaque marker encapsulated between the outer and inner materials.

14. The expandable stent of claim 6, wherein the inner material has a thickness between about 0.1 mm and about 0.15 mm.

15. The expandable stent of claim 6, wherein the outer material has a thickness of one of: between about 0.05 mm and about 0.07 mm, between about 0.07 mm and about 0.20 mm, between about 0.20 mm and about 0.60 mm, and between about 0.40 mm and about 0.60 mm.

16. The expandable stent of claim 6, wherein the outer material comprises a woven material formed from yarns having at least one of: a linear density between about 20 decitex and about 25 decitex, and a thread density between about 160 picks per inch and about 225 picks per inch.

17. The expandable stent of claim 16, wherein the outer material is configured to experience a reduction in thickness of up to between about 15% and about 20% under compression in a deployed condition of the expandable stent.

18. The expandable stent of claim 6, wherein the outer material has a stretchability between about 40% and about 60%.

19. The expandable stent of claim 6, wherein the outer material has a stretchability between about 60% and about 100% in both an axial direction and a circumferential direction.

20. An expandable stent comprising:
an expandable frame extending axially from a proximal end to a distal end;
an outer material attached to an exterior of the expandable frame to define an outer periphery of the expandable stent; and
an inner material attached to an interior of the expandable frame to define an inner periphery of the expandable stent,
wherein at least a portion of the outer material is axially aligned with at least a portion of the inner material, and wherein the axially aligned portions of the outer material and the inner material encapsulate at least a portion of the expandable frame.
